(19) United States
(12) Patent Application Publication
(10) Pub. No.: US 2010/0312054 A1
(43) Pub. Date: Dec. 9, 2010

(54) PROSTATIC TISSUE REMOVAL AND/OR PROSTATIC CAPSULOTOMY FOR TREATMENT OF CONDITIONS

(75) Inventors: Mordechay Beyar, Caesarea (IL); Oren Globerman, Kefar-Shemaryahu (IL); Benad Goldwasser, Tel-Aviv (IL); Hila Wachsler-Avrahami, Tel-Aviv (IL); Ofer Nativ, Rishon Le'zion (IL); Ronen Shavit, Tel-Aviv (IL)

Correspondence Address: EDWARDS ANGELL PALMER & DODGE LLP, P.O. BOX 55874, BOSTON, MA, 02205, US

(73) Assignee: N.M.B. Medical Applications Ltd., Herzeliya (IL)

(21) Appl. No.: 12/795,199

(22) Filed: Jun. 7, 2010

Related U.S. Application Data
(63) Continuation-in-part of application No. PCT/IL2008/001586, filed on Dec. 7, 2008.
(60) Provisional application No. 61/050,628, filed on May 6, 2008.
(60) Provisional application No. 61/080,258, filed on Jul. 13, 2008.
(60) Provisional application No. 61/185,660, filed on Jun. 10, 2009.
(60) Provisional application No. 61/213,732, filed on Jul. 8, 2009.

(51) Int. Cl. A61B 1/018; A61B 18/22

(52) U.S. Cl. 600/108; 606/10

(57) ABSTRACT

Apparatus and methods are provided including identifying a subject as suffering from a disorder of an organ of the subject. In response to the identifying, a shaft is inserted into the organ, not via a natural lumen of the subject. Using a laser beam emitted from a distal portion of the shaft, a portion of the organ is ablated. A vicinity of the portion of the organ is cooled by introducing a fluid to the vicinity, and, subsequently aspirating the fluid therefrom, the introduction and aspiration of the fluid being performed via the shaft, while the shaft is inside the organ. Other embodiments are also described.

### CROSS-REFERENCES TO RELATED APPLICATIONS

The present application:

(i) is a continuation-in-part of PCT Application PCT/IL2008/001586, filed Dec. 7, 2008, entitled “Prostatic capsulotomy for treatment of conditions,” to Goldwasser, which claims the benefit of (a) U.S. Provisional Patent Application 61/005,637 to Goldwasser, filed Dec. 5, 2007, (b) U.S. Provisional Patent Application 61/050,628 to Goldwasser, filed May 6, 2008, and (c) US Provisional Patent Application 61/080,258 to Goldwasser, filed Jul. 13, 2008, all of which applications are incorporated herein by reference; and

(ii) claims the benefit of:
(a) U.S. Provisional Patent Application 61/185,660 to Goldwasser, filed Jun. 10, 2009, which is incorporated herein by reference; and
(b) U.S. Provisional Patent Application 61/213,732 to Beyar, filed Jul. 8, 2009, which is incorporated herein by reference.

### FIELD OF EMBODIMENTS OF THE INVENTION

Some applications of the present invention generally relate to medical apparatus and methods for treating the prostate. Specifically, some applications of the present invention relate to medical apparatus and methods for the treatment of benign prostatic hyperplasia, focal prostatic cancer, and/or other conditions.

### BACKGROUND

Benign prostatic hyperplasia is a condition in which the prostate enlarges to the point where it constricts the urethra and impedes the flow of urine, making urination difficult and painful, and in extreme cases completely impossible.

Valleylab (Colorado, USA) manufactures the following products:
LigaSure™ V, which is described as being capable of performing sealing and cutting in laparoscopic procedures;
LigaSure Atlas™, which is described as being for use in laparoscopic surgery;
The LigaSure Atlas™ is described as being capable of grasping and holding sealed tissue for easy transection; and
LigaSure™ Lap, which is described as a laparoscopic 5 mm instrument for sealing and fine dissection of structures in small surgical spaces.

InterVentional Technologies Inc. (CA, USA) manufactures The Cutting Balloon®.

Covidien (USA) manufactures the SPACEMAKER™ PLUS Dissector System, which is described as a dissection balloon that is available in 2 shapes (round or oval).

Johnson and Johnson (NJ, USA) manufactures the Harmonic Scalpel®, which is described as an ultrasonic cutting and coagulating surgical device.

Insightec (Tirat Carmel, Israel) manufactures the Exablate® device, which uses MR guided focused ultrasound.

Medtronic (US) manufactures the Straightshot® M4 Microdebrider.

The following references may be of interest:
U.S. Pat. No. 5,277,696 to Hagen
U.S. Pat. No. 5,312,392 to Hofstetter
U.S. Pat. No. 5,458,612 to Chin
U.S. Pat. No. 5,794,626 to Kieturakis
U.S. Pat. No. 6,030,400 to Johnson
U.S. Pat. No. 6,136,014 to Sirimanne
U.S. Pat. No. 6,190,381 to Olsen et al.
U.S. Pat. No. 6,491,672 to Slepian et al.
U.S. Pat. No. 6,602,265 to Dubrul et al.
U.S. Pat. No. 6,623,437 to Hinchliffe et al.
U.S. Pat. No. 6,979,332 to Adams et al.
U.S. Pat. No. 7,229,413 to Violante et al
U.S. Pat. No. 7,300,447 to Eliachar
US 2009/0062793 to Eliachar
US 2009/0024138 to Saleh
US 2008/0009891 to Cohn
US 2007/0100209 to Takahashi
US 2007/0162136 to O'Neil
US 2007/0089293 to Pitzele
US 2006/0135882 to Bleich
US 2004/0199194 to Witt
US 2004/0102770 to Goble
US 2007/0203517 to Williams
US 2006/0276871 to Lamson
US 2006/0184188 to Li
PCT WO 01/072200 to Yossepowitch
PCT WO 03/096912 to Yossepowitch
PCT WO 05/079682 to Yossepowitch
PCT WO 07/000,754 to Eliachar
PCT WO 07/031,990 to Eliachar
PCT WO 07/088,533 to Eliachar
“Cervico-prostatic incision in the treatment of benign prostatic hypertrophy. Apropos of 40 cases,” by Njeh et al., J Urol (Paris), 1991; 97(3):137-9
“Clinical observation: extraurethral prostatectomy by ligating prostatic artery,” by Cao et al., BJU International (2002), 90(Suppl. 2), 160
“Closed Prostatic Commissurotomy: An Endoscopic Technique for the Treatment of Benign Prostatic Hypertrophy,” by Shafik, British Journal of Urology (1988), 62, 431-433
“Endoscopic perineal approach to the presacral space: a feasibility study,” by Ganger et al., J Surg Endosc 2008 June; 22:1987-91
“Dissection of prostate coalesce in treatment of benign prostate hypertrophy with relatively small volume,” by Qiu et al., Zhonghua Yi Xue Za Zhi, 2007-July, Vol 87 (issue 26): pp 1852-3
“The Prostate,” by David F. Paulson, Genitourinary Surgery, Churchill Livingstone, 1984, pp 313-398
“Initial experience with extraperitoneal endoscopic radical retropubic prostatectomy,” by Raboy et al., Urology 1997 December; 50(6):849-53
“In-Vitro Visualization of Biopsy Needles with Ultrasound: A Comparative Study of Standard and Echogenic Needles Using an Ultrasound Phantom,” by Hopkins R. E. et al., Clin Radiol 2001; 56: 499-502; and
“New trends in the management of benign prostatic hyperplasia,” Dr. Safdar H. Javed Sial, THE PROFESSIONAL VOL. 02, NO. 03 July, August, September 1995
“Point of view: Capsular influence on benign prostatic hyperplasia,” by F. Hinman Jr., Urology, October 1986, Vol. XXVIII, Number 4, pp 347-350
“PROSTATE ENLARGEMENT (BPH): Perineal Prostatectomy,” published by The Hutchinson Clinic, as an article in “The A to Z of patient information,” published by the clinic
“Radical perineal prostatectomy: cost efficient, outcome effective, minimally invasive prostate cancer management,” by Harris, Eur Urol. 2003 September; 44(3):303-8
“Surgical case report: Prostatic omentalization,” by Anderson et al., DVM Pulse, November 2001
“The enlarged prostate: a brief history of its surgical treatment,” by H. W. Herr, 2006 BJU International, 98, 947-952
“Transperineal microwave thermoablation in patients with obstructive benign prostatic hyperplasia: a phase I clinical study with a new mini-choked microwave applicator,” by Bartoletti et al., J Endourol. 2008 July; 22(7):1509-17
“Transrectal ultrasound guided manipulation of the canine prostate with minimal intervention,” by Chang, Laboratory animals (1997) 31, 219-224

### SUMMARY OF EMBODIMENTS

For some applications of the present invention, a subject is identified as suffering from benign prostatic hyperplasia (BPH), and typically (but not necessarily) has not been diagnosed with prostate cancer. For some applications, the subject is identified as having a prostatic urethra obstruction that is not based on BPH, and the obstruction is treated (i.e., reduced, or removed), using the methods and apparatus described herein. An instrument incises the prostatic capsule surrounding the prostate, to relieve pressure in the prostate. Following the incising, the prostate is more free to expand, for example, in response to the presence and/or growth of the BPH, reducing the pressure that the BPH exerts on the urethra. Typically, the pressure is relieved without removing the BPH itself, or for some applications, without even touching the BPH. For some applications, a portion of the prostate may be cut or removed, as described herein.

Typically, a physician makes one or more incisions in the prostatic capsule, e.g., between two and eight incisions. For some applications, the incisions in the prostatic capsule are in the superior-inferior direction or in the medial-lateral direction. Alternatively, the physician incises and/or excises the prostatic capsule, in whole or in part, using, for example, laser-induced vaporization of the capsule, radiofrequency (RF) energy, or a cold knife.

Typically, an imaging device (e.g., an endoscopic imaging device, an ultrasound device, a CT imaging device, an MRI imaging device, a cystourethroscope, or a laparoscope) is used by the physician to facilitate the passage of the instrument to the prostatic capsule.

For some applications of the present invention, the instrument gains access to the capsule via passage through the abdominal wall. In another application, the instrument accesses the capsule via passage through the urethra. In yet another application, the instrument gains access to the prostatic capsule via passage through the perineum.

For some applications, access of the instrument to the prostatic capsule is aided by the inflation of a balloon at the distal end of a longitudinal surgical introduction tool, such as a trocar, a delivery catheter, or an endoscope, that places the instrument inside or outside the capsule, in the vicinity of the capsule. For some applications of the present invention, inflation of the balloon pushes the prostate in the posterior direction and/or pushes the urinary bladder in the superior direction. On deflation of the balloon, the space where the balloon had been inflated is insufflated with air or carbon dioxide, to provide additional room for the instrument to gain access to the prostatic capsule. As appropriate, the balloon may surround the distal end of the introduction tool, or be passed through a channel of the introduction tool, for example, through a channel of an endoscope.

For some applications, the instrument incises the prostatic capsule from within the capsule, gaining access to the capsule transurethrally, transperineally, or transabdominally.

For some applications, a balloon device is used for treating BPH, the balloon device including at least one incision or excision element designed to incise or excise the prostate capsule, with or without incision/excision of the prostate tissue.

For some applications, the balloon is filled with a compressible and/or a non-compressible fluid, such as air, carbon dioxide, or water, to create a space near the prostate, thus facilitating the procedure. The space may be, for example, between the anterior surface of the prostate and the posterior surface of the pubic bone.

For some applications, following balloon expansion, the balloon is attached to the prostate, and the prostate capsule is incised and/or excised at one or more locations.

For some applications, the incision/excision elements (“cutting elements”) are capable of incising or excising soft tissue, such as the prostate capsule and the underlying prostatic tissue. The cutting method may include use of, for example, a knife, a sharp edge, diathermia, a haimonic scalpel (e.g., a Harmonic Scalpel® manufactured by Johnson and Johnson), a monopolar or bipolar device (e.g., a LigaSure™ device manufactured by Valleylab), or a laser, such as a green laser.

For some applications, two cutting elements are incorporated into the balloon. Optionally, the balloon comprises more cutting elements, for example, three, four, or a greater number of such elements. For some applications of the invention, longitudinal cutting elements are used. For some applications, the cutting elements are parallel to each other. For some applications, two or more parallel cutting elements are spaced from each other by a distance of between 10 mm and 100 mm, for example, between 20 mm and 70 mm, or between 45 and 55 mm. Alternatively, the parallel cutting elements are spaced from each other by a different distance. For some applications, two cutting elements are oriented such that the cutting elements incise and/or excise the prostate and/or prostate capsule at the 2 o'clock and 10 o'clock positions (the pubis defining the 12 o'clock position, as is known in the art). Alternatively or additionally, the cutting elements are oriented or spaced differently with respect to each other, and/or, in general, in order to facilitate the creation of a different cutting pattern along the prostate capsule.

For some applications, the cutting element(s) are mounted on a wire or similar construction, and are movable, to allow their movement along a predefined path.

For some applications of the invention, the balloon has a rectangular shape. Alternatively, the balloon may be shaped differently, for example, the balloon may be spherical, oval, trapezoid, square, or a different shape.

For some applications, the balloon has a width and/or length of between 20 mm and 70 mm, for example, between 30 mm and 60 mm, or between 40 mm and 50 mm. Alternatively the balloon has different dimensions.

For some applications, the balloon is guided by an imaging device, for example, an endoscopic imaging device, an ultrasound imaging device (e.g., a transrectal or a transabdominal ultrasound probe), a CT imaging device, and/or an MRI imaging device. For some applications, the imaging device and balloon are introduced into the subject body via a single access port. Alternatively, different access ports are used for the introduction of the balloon and the imaging device.

For some applications, a single port access imaging device is used, for example, for treating BPH. For some applications, the single port access imaging device allows the surgeon to complete an entire procedure through a single access point, located, for example, near the umbilicus or at a small distance from it, for example, 10 mm to 30 mm from the umbilicus, or at a location between the upper border of the pubic symphysis and the umbilicus.

For some applications, a single port access imaging device, for instance an endoscope, comprises:

a lighting source, such as a white LED;

a camera; and

a working channel, through which a soft tissue cutting device, such as a diathermia device, a harmonic scalpel (e.g., the Harmonic Scalpel® manufactured by Johnson and Johnson), a monopolar or bipolar device (e.g., a LigaSure™ device manufactured by Valleylab), a laser (e.g., green laser) device, and or a scalpel is introduced and used.

For some applications, the endoscope contains additional lumens, for example, a lumen that is used for the introduction of air and/or another fluid, the air or other fluid being utilized for camera lens cleaning, and/or for inflating a space such as one created between the prostate and pubic symphysis.

For some applications, the endoscope body is made of metal. Alternatively, the endoscope body may be made of polymeric rigid construct. For some applications, the endoscope includes a multi-lumen endoscope, as is known in the art.

For some applications, the imaging device is a single use instrument. Alternatively, the imaging device is reusable.

For some applications, an access needle that also serves as a working channel is used, for example, for the treatment of BPH.

For some applications of the invention, the needle is used with a stylet, to facilitate insertion of the needle through soft tissue. For some applications of the invention, a blunt-edged stylet is used.

For some applications of the invention, the needle and/or stylet are manufactured from a relatively flexible material, for example, a metal such as Nickel-Titanium (“nitinol”), or a polymer. Alternatively, the needle is shaped to be curved. For some applications, the flexibility and/or the shape of the needle facilitates insertion of the needle in a curved or straight path, or a path that includes a curved portion and a straight portion. For example, the needle, and/or another instrument, may be inserted along a path that closely follows the pubic symphysis towards the prostate (this being an application of a retropubic approach).

For some applications of the invention, the needle outer diameter is less than 1 mm, 2 mm, 3 mm, 4 mm, 5 mm, 6 mm, 7 mm, 8 mm, 9 mm, or 10 mm. Alternatively, the needle has different dimensions.

For some applications of the present invention, following positioning of the needle, the stylet is removed and a cutting instrument is introduced via the needle, to allow incision(s) or excision(s) of the prostate capsule. The cutting instrument may comprise, for example, a stylet with a cutting element at its distal tip, and may be made of a flexible material, for example, a metal, such as nitinol, and/or a polymeric material. For some applications, prior to the introduction of the cutting instrument, a balloon device is inserted via the needle and inflated, to create a space in a vicinity of the prostate.

For some applications, the cutting instrument is curved or bent. For example, the cutting instrument may be curved and/or bent in one or more locations. For some applications, the cutting instrument has a curved arc having a length of between about 20 mm and 100 mm. For some applications, the shape of the cutting instrument facilitates insertion of the cutting instrument, such that the cutting instrument follows the outer surface of the prostate during the insertion. For some applications, the cutting instrument has a bent cutting element at its distal end, located, for example, 2 mm to 10 mm from the device distal tip, that provides for the cutting function. The bent portion may be bent with respect to the longitudinal axis of a portion of the device immediately proximal to the bent portion by an angle of between 10 degrees and 20 degrees, for example, about 15 degrees.

For some applications of the invention, the cutting element may include a knife, a scalpel, a diathermia device, a harmonic scalpel (e.g., the Harmonic Scalpel® manufactured by Johnson and Johnson), a monopolar or bipolar device (e.g., a LigaSure™ device manufactured by Valleylab), a laser (e.g., a green laser), and/or different device for cutting soft tissue that is known in the art.

In accordance with respective applications of the invention, the distal tip of the cutting instrument has a sharp tip or a wide tip, the tip having a width of 2 mm to 15 mm, for example. For some applications, the cutting tip is made of a nitinol core, covered with a polymer layer having a thickness of 0.02 mm to 2 mm.

For some applications of the present invention, a limiting structure/device is incorporated onto the cutting instrument to limit the penetration of the cutting element to a pre-defined depth. For some applications, the limiting device is a small balloon device or a pulley device.

For some applications of the present invention, an imaging device, such as an endoscope, ultrasound, CT or MRI imaging device, is used in combination with the access needle, to guide the needle positioning and incision and/or excision of the prostate capsule. For some applications of the invention, a transrectal or transabdominal ultrasound imaging device is used.

For some applications of the invention, a balloon is used and filled with a compressible fluid, a non-compressible fluid, and/or with an ultrasound opaque substance to create a working space near the prostate. For some applications, the balloon is introduced via the access needle. The balloon may have, for example, a round shape or a rectangular shape.

For some applications of the invention, more than one access needle is introduced, for example, two access needles are introduced, optionally, parallel to each other, and the cutting procedure is performed via the two ports.

For some applications of the invention, an access stylet and/or the cutting instrument have non-round cross sections, for example square, triangular, or rectangular-shaped cross sections, in order to facilitate accurate orientation of these devices relative to the prostate outer surface.

For some applications, following incision and/or excision (“cuts”) of the prostate capsule for the treatment of BPH, a method and apparatus are used to prevent the cuts from re-closing.

For some applications of the invention, a device is implanted around at least a portion of the circumference of the prostate capsule, to cover the cuts, following incision(s)/excision(s) of the prostate capsule and/or prostate tissue. In alternative applications of the present invention, the device is implanted without previous incision of the prostate capsule or prostate tissue.

For some applications of the invention, the device(s) may be implanted so that it is anchored in deeper prostate tissue. For some applications, the device has an omega shape.

For some applications of the present invention, the device is flexible (e.g., made of nitinol), and is configured such that following its implantation it works as a spring to preserve the cuts and prevent their closure.

For some applications of the present invention, the spring-like device exerts continuous radial force against the prostate tissue to reduce the constrictive force on the prostate urethra and/or bladder neck.

For some applications of the present invention, one or more spring-like devices are placed along each cut of the prostate capsule to prevent the cuts from re-closing. For example, three flexible devices may be implanted along each cut.

For some applications of the invention, the device(s) is implanted via the access needle described hereinabove. For some applications, the implantation is guided by an imaging device.

For some applications of the invention, subsequent to accessing the prostate, for example, transperineally or transabdominally (e.g., a retropubic approach), at least a portion of the prostate tissue is excised in order to relieve pressure in the prostate, without damaging the urethra. For some applications, the prostate capsule is also incised or excised. Alternatively, the prostate capsule remains intact (except for a small access port to the prostate), and only prostate tissue is removed.

For some applications, an imaging device is used to enable visualization of the procedure. For some applications, the imaging device includes at least one imaging device selected from the group consisting of: an endoscopic imaging device, an ultrasound device, such as a transrectal ultrasound device, a CT imaging device, an MRI imaging device, a cystourethroscope, and a laparoscope.

For some applications of the invention, the removal of prostate tissue is performed with at least one instrument selected from the group consisting of: a cold knife, a cauterizing tool, a laser, a morcellation device, and an ablation device. Examples of such devices include: a diathermia device, a harmonic scalpel (e.g., as manufactured by Johnson and Johnson), a monopolar or bipolar device (e.g., a LigaSure™ device manufactured by Valleylab), a green laser device, a red laser device, a laser device the laser having a wavelength in the range of 500 nm to 2100 nm, and/or a plasma device.

For some applications of the invention, incision and/or excision of the prostatic capsule is performed extraurethrally, from within the prostate. For some applications of the invention, access into the prostate is gained, for example, perineally. For some applications, an instrument used to cut the prostate capsule is positioned at the anterior portion of the prostate, to cut the capsule from inside the prostate. Alternatively, a perineal approach is used to cut the prostate capsule, for example, at its anterior portion, from outside of the capsule.

For some applications, an imaging device is used to enable visualization of the procedure. For some applications, the imaging device includes at least one imaging device selected from the group consisting of: an endoscopic imaging device, an ultrasound device, such as transrectal ultrasound, a CT imaging device, an MRI imaging device, a cystourethroscope, and a laparoscope.

For some applications of the invention, incision and/or excision of the prostatic capsule may be performed with at least one instrument selected from the group consisting of: a cold knife, a cauterizing tool, a laser, a morcellation device, and an ablation device. Examples of such devices include a diathermia device, a harmonic scalpel (e.g., the Harmonic Scalpel® manufactured by Johnson and Johnson), a monopolar or bipolar device (e.g., a LigaSure™ device manufactured by Valleylab), a green laser device, a red laser device, a laser device the laser having a wavelength in the range of 500 nm to 2100 nm, and/or a plasma device. For some applications of the invention, a device capable of tissue cutting and coagulating is used.

For some applications of the invention, a cutting device, including a pair of clips and a cutting element (such as scissors or blade) in between the clips, is used for prostate capsule incision. Using the clips, the tissue of the prostate capsule is grasped in two locations, and capsule incision is performed between these clips, by the cutting element.

For some applications of the invention, one or more of the procedures described herein is used to treat prostate cancer, for example, when a focal malignant growth is diagnosed. In such cases, the cancerous tissue is removed extraurethrally (i.e., without urethral access), using one of the methods described herein.

For some applications, the procedures described herein are computer-aided and/or robot-aided. For example, introduction and/or operation of the instrumentation may be computer-aided and/or robot-aided.

For some applications of the invention, treating BPH includes performing at least one incision on the prostate capsule, and/or performing prostate tissue resection. For some applications of the invention, the prostate gland is approached percutaneously, transperineally, and/or via other approaches (e.g., an abdominal approach, such as a retropubic and/or a suprapubic approach, and/or a transurethral approach).

For some applications, the procedure is performed under direct vision, such as under the vision of an endoscope. For some applications, the visualization means are introduced through the perineum into the retropubic space, between the pubic bone and the prostate. Alternatively, the visualization means are introduced via the perineum to be positioned in a different location, for example, posteriorly to the prostate gland (i.e., between the prostate and the rectum). For some applications, the visualization means are introduced abdominally. For some applications, additional visualization means may be used, such as ultrasound (e.g., transrectal ultrasound), MRI or CT imaging device.

For some applications, a single port access imaging device (e.g., an endoscope) is used to allow performance of the entire procedure via a single access port (i.e., a single incision in the skin), located, for example, at the perineum urogenital triangle. Alternatively, an imaging device is inserted via two or three ports, the ports being located, for example, at the perineum urogenital triangle. For some applications, the ports are used to facilitate access of a light source of the imaging device, a camera, a working channel through which apparatus to treat the prostate is introduced (e.g., a cutting device, a diathermia device, a laser device, etc.), and/or one or more additional lumens. For example, an additional lumen may be used for the introduction of air (or a different fluid) for creation of a space near the prostate, and/or cleaning of the camera lens. For some applications, the endoscopic cannula is relatively rigid, for example, in order to facilitate introduction of an instrument used to incise and/or excise the prostate tissue and/or the prostatic capsule. Alternatively, a relatively flexible endoscopic cannula is used, for example, in order to facilitate manipulation and placement of an instrument introduced through the cannula.

For some applications, introduction of a direct vision instrument (e.g., an endoscope) into the retropubic space via a perineal approach, rather than via a laparoscopic approach (i.e., transabdominally), does not require (a) substantial inflation of the retropubic space, (b) dissection of the abdominal wall, and/or the peritoneum, or (c) inflation of the abdominal space. For some applications, it is desirable not to inflate the abdominal space, since this may require endotracheal intubation, which may have undesirable effects on the subject's body.

For some applications of the invention, a balloon is inserted and inflated adjacent to the prostate, to create a space near the prostate and thus facilitate the procedure (for example, to enlarge the retropubic space between the prostate and the pubic bone). Optionally, a single balloon is used to create space between the prostate and the pubic bone. Alternatively, two, smaller balloons are used, and inflated in the right and left portions of the retropubic space, respectively. For some applications, during such a procedure, no direct contact is made with the puboprostatic ligament. For some applications, the balloon is designed to have a configuration and dimensions that comply with the anatomy of its designated location. For some applications, the balloon is introduced into the patient body percutaneously, through a cannula. Optionally, the endoscopic device comprises a lumen through which balloon is introduced. For some applications, the balloon is inflated by filling it with a fluid, such as water, air, and/or carbon dioxide.

For some applications of the invention, incision and/or excision (e.g., resection) of the prostate tissue (optionally, including prostate capsule) may be performed by using, for example, one or more of the following devices: a diathermia device, a cold knife, a cauterizing tool, monopolar and bipolar RF devices, a laser device, a morcellation device, a microdebrider, a cryotherapy device, and an ablation device. Alternatively or additionally, a device that includes a tissue collection member (to collect and remove the resected tissue following its resection, e.g., as described herein) is used.

For some applications, cutting and/or resection of the tissue includes coagulation (sealing of blood vessels).

For some applications, cutting and/or resecting the prostate capsule and/or the prostate is performed from the outside of the prostate. For example, a cutting and/or resecting device may be introduced, transperineally, into the retropubic space, and then incise and/or excise prostatic tissue from outside the anterior side of the prostate. Alternatively or additionally, a cutting and/or resecting device is introduced into the prostate, for example, transperineally. For example, the device may be inserted into the anterior part of the prostate, or to the right lobe and/or the left lobe of the prostate, to resect prostate tissue and/or to cut the prostatic capsule from within the prostate.

There is therefore provided, in accordance with some applications of the present invention, apparatus, including:

an elongate element for inserting into a subject's body;

a joint at a distal end of the elongate element;

an arm having a proximal end and a distal end, the distal end of the articulatable at the joint with the elongate element, wherein in a closed position of the arm with respect to the elongate element, a portion of the arm faces a portion of the elongate element; and

a cutting element coupled to at least one of the portions.

For some applications, the cutting element is slidably coupled to the elongate element.

For some applications, the cutting element includes a cutting element selected from the group consisting of: a knife, a sharp edge, a diathermia device, a harmonic scalpel, an RF device, a plasma device, and a laser.

For some applications, the cutting element includes one or more blades disposed on the arm.

For some applications, the joint includes a plastic hinge

For some applications, the cutting element is configured to sequentially cut tissue of the subject at successively more proximal cutting sites, by the distal end of the elongate element being moved to the successively more proximal cutting sites.

For some applications, the apparatus further includes two balloons disposed on opposing sides of the elongate element at the distal end of the elongate element, the balloons being configured to create a space distal to the balloons and distal to the elongate element, by being inflated.

For some applications, the apparatus further includes at least one coagulation electrode configured to coagulate tissue of the subject.

For some applications, the coagulation electrode includes two or more bipolar coagulation electrodes.

For some applications, the coagulation electrode includes a monopolar coagulation electrode.

For some applications, the cutting element includes a coagulation electrode configured to coagulate tissue of the subject.

For some applications, the cutting element further includes a mechanical cutting element.

For some applications, the coagulation electrode includes two or more bipolar coagulation electrodes.

For some applications, the coagulation electrode includes one monopolar coagulation electrode.

For some applications, the electrode is coated with a coating produced by an alodine process and a hard anodization process.

For some applications, the coating has a thickness of less than 100 microns.

For some applications, the arm includes a penetration element configured to penetrate tissue of the subject.

For some applications, the penetration element is disposed at the proximal end of the arm.

For some applications, the penetration element includes a sharp edge.

For some applications, the arm is configured to grasp tissue of the subject by being at least partially closed with respect to the elongate element.

For some applications, the cutting element is configured to cut the grasped tissue, and the apparatus further includes a coagulation electrode configured to coagulate the grasped tissue.

For some applications, the cutting element is configured to cut the grasped tissue.

For some applications, the cutting element is configured to cut and coagulate the grasped tissue.

For some applications, the distal end of the elongate element is configured to be inserted to a vicinity of an anterior side of a prostate of the subject, and the cutting element is configured to cut a prostate capsule of the prostate.

For some applications, the distal end of the elongate element is configured to be inserted to inside the subject's prostate, and the cutting element is configured to cut the prostate capsule from inside the prostate.

For some applications, the distal end of the elongate element is configured to be inserted to outside the subject's prostate, and the cutting element is configured to cut the prostate capsule from outside the prostate.

For some applications, the elongate element is configured to be transperineally inserted to the vicinity of the anterior side of the subject's prostate.

For some applications, the elongate element is configured to be transabdominally inserted to the vicinity of the anterior side of the subject's prostate.

For some applications, the elongate element is shaped to define a curved distal region and a straight proximal region of the elongate element, the curved shape of the distal region of the elongate element facilitating retropubic insertion of the distal end of the elongate element.

There is further provided, in accordance with some applications of the present invention, apparatus, including:

an elongate element for inserting into a subject's body, the elongate element having a proximal-facing portion at a distal end of the elongate element,

the proximal-facing portion including a cutting element, the cutting element configured to sequentially cut tissue of the subject at successively more proximal cutting sites, by the distal end of the elongate element being moved to the successively more proximal cutting sites.

For some applications, the cutting element includes a cutting element selected from the group consisting of: a knife, a sharp edge, a diathermia device, a harmonic scalpel, an RF device, a plasma device, and a laser.

For some applications, the apparatus further includes two balloons disposed on opposing sides of the elongate element at the distal end of the elongate element, the balloons being configured to create a space distal to the balloons and distal to the elongate element, by being inflated.

For some applications, the cutting element is a sharp tip of the proximal-facing portion.

For some applications, the proximal-facing portion is a continuation of the elongate element.

For some applications, the proximal-facing portion and the elongate element are formed as one integral body, in which the proximal-facing portion includes a curved aspect that makes the proximal-facing portion face in a proximal direction.

For some applications, the cutting element includes an electrode and a mechanical cutting element, the cutting element being configured to cut the tissue by applying pressure to the tissue, and to coagulate the tissue by driving a current into the tissue.

For some applications, at least a portion of the proximal-facing portion includes a shape memory material.

For some applications, the apparatus further includes an elongate-element introduction-device that defines a lumen, the elongate element is inserted via the lumen, and the proximal-facing portion is configured to open with respect to the elongate element, due to the shape memory material, when the proximal-facing portion passes out of a distal end of the lumen.

There is additionally provided, in accordance with some applications of the present invention, apparatus for treating an obstruction of a prostatic urethra of a subject, including:

a balloon that during inflation of the balloon is configured to expand in a first direction by more than 200% of an expansion of the balloon in each of second and third directions, the first, second and third directions being mutually perpendicular to each other;

one or more cutting elements coupled to a surface of the balloon, at least a portion of the surface being disposed in a plane that is defined by the second and third directions;

an elongate element configured to place the balloon in a vicinity of an anterior side of a prostate of the subject; and

a balloon control unit configured to create a space in the vicinity by inflating the balloon.

For some applications, during inflation of the balloon, the balloon is configured to expand in the first direction by more than 400% of the expansion of the balloon in each of the second and third directions.

For some applications, the one or more cutting elements protrude from the surface.

For some applications, the cutting elements include a cutting element selected from the group consisting of: a knife, a sharp edge, a diathermia device, a harmonic scalpel, an RF device, a plasma device, and a laser.

For some applications, the length of the balloon in the second and third directions is less than 60 mm, when the balloon is maximally inflated.

For some applications, the cutting elements include one or more cutting elements that are substantially mobile with respect to the surface of the balloon in the plane that is defined by the second and third directions.

For some applications, the cutting elements include one or more cutting elements that are substantially immobile with respect to the surface of the balloon in the plane that is defined by the second and third directions.

For some applications, the balloon is configured to be in a folded disposition during insertion of the balloon into the vicinity, and each of the cutting elements are disposed on a respective fold of the folded balloon.

For some applications, an outer surface of the balloon is shaped to match a shape of the anterior side of the prostate.

For some applications, the outer surface of the balloon is shaped concavely when maximally inflated.

There is further provided, in accordance with some applications of the present invention, a method, including:

inserting a tool into a subject's body via a perineum of the subject; and

while the tool is inside the subject's body, performing minimally invasive surgery on the subject using the tool.

There is further provided, in accordance with some applications of the present invention, a method for treating an obstruction of a prostatic urethra of a subject, including:

identifying that the subject has a constricted urethra due to the prostatic urethra obstruction; and

relieving pressure on the urethra caused by the prostatic urethra obstruction, by performing an action on at least a portion of a capsule of a prostate of the subject, the action being selected from the group consisting of: incising, and excising.

For some applications, performing the selected action includes grasping the portion and cutting the grasped portion.

For some applications, relieving pressure on the urethra includes performing at least one surgical access selected from the group consisting of:

transabdominally accessing the capsule,

transperineally accessing the capsule,

transurethrally accessing the capsule, and

non-transurethrally accessing the capsule.

For some applications, relieving pressure on the urethra includes performing at least one surgical access selected from the group consisting of:

transabdominally accessing the capsule, and

transperineally accessing the capsule,

For some applications, relieving pressure on the urethra includes performing the selected action on at least the portion of the capsule from a site within the prostate.

For some applications, performing the selected action on at least the portion of the capsule includes performing the selected action on at least the portion of the capsule with at least one instrument selected from the group consisting of: a cold knife, a cauterizing tool, and a laser.

For some applications, performing the selected action on at least the portion of the capsule includes performing the selected action on at least the portion of the capsule with at least one instrument selected from the group consisting of: a sharp edge, a diathermia device, a harmonic scalpel, and an RF device.

For some applications, performing the selected action on at least the portion of the capsule includes forming at least one incision in a superior-inferior direction in the prostatic capsule.

For some applications, performing the selected action on at least the portion of the capsule includes forming at least one incision in a medial-lateral direction in the prostatic capsule.

For some applications, performing the selected action on at least the portion of the capsule includes excising the prostatic capsule.

For some applications, performing the selected action on at least the portion of the capsule includes incising the prostatic capsule.

For some applications, the method further includes inserting a cutting element into the subject's body, and performing the selected action on the portion of the capsule includes sequentially cutting the capsule at successively more proximal cutting sites by moving the cutting element to the successively more proximal cutting sites.

For some applications, the method further includes:

inserting a balloon into a body of the subject, to a vicinity of an anterior side of the subject's prostate;

creating space in the vicinity by inflating the balloon such that the balloon expands in a first direction by more than 200% of an expansion of the balloon in each of second and third directions, the first, second and third directions being mutually perpendicular to each other; and

performing the selected action on the portion of the capsule includes cutting the capsule with one or more cutting elements that are coupled to a surface of the balloon, the surface being disposed at least partially in a plane that is defined by the second and third directions.

For some applications, performing the selected action on the portion of the capsule includes moving the cutting elements with respect to the surface of the balloon in the plane that is defined by the second and third directions.

For some applications, performing the selected action on the portion of the capsule includes substantially not moving the surface of the balloon during performance of the selected action.

For some applications, performing the selected action on the portion of the capsule includes moving the balloon with respect to the capsule.

For some applications, performing the selected action on the portion of the capsule includes, during the performance of the selected action, substantially not moving the cutting elements with respect to the surface of the balloon in the plane that is defined by the second and third directions.

For some applications, performing the selected action on at least the portion of the capsule includes forming a plurality of incisions in the prostatic capsule.

For some applications, performing the selected action on at least the portion of the capsule includes forming 1-3 incisions in the capsule.

For some applications, performing the selected action on at least the portion of the capsule includes forming 4-10 incisions in the capsule.

For some applications, performing the selected action on at least the portion of the capsule includes forming a plurality of incisions in the prostatic capsule in different directions.

There is additionally provided, in accordance with some applications of the present invention, apparatus for treating an obstruction of a prostatic urethra of a subject, including:

an instrument, configured to perform an action on at least a portion of a capsule of a prostate of the subject to an extent that is sufficient to relieve pressure in the prostate due to the prostatic urethra obstruction, the action being selected from the group consisting of: incising, and excising; and

an imaging device, configured to facilitate guiding of the instrument to the prostatic capsule of the subject.

For some applications, the imaging device includes at least one imaging device selected from the group consisting of: an endoscopic imaging device, an ultrasound device, a CT imaging device, an MRI imaging device, a cystourethroscope, and a laparoscope.

For some applications, the instrument is configured to access the capsule via passage through at least one site selected from the group consisting of: an abdominal wall of the subject, a urethra of the subject, and a perineum of the subject.

For some applications, the instrument includes at least one instrument selected from the group consisting of: a cold knife, a cauterizing tool, and a laser.

For some applications, the instrument is configured to make at least one incision in a superior-inferior direction, in the prostatic capsule.

For some applications, the instrument is configured to make at least one incision in a medial-lateral direction, in the prostatic capsule.

For some applications, the instrument is configured to make a plurality of incisions in the prostatic capsule.

For some applications, the instrument is configured to make a plurality of incisions, at least some of which are in different directions.

For some applications, the instrument is configured to excise at least a portion of the prostatic capsule.

For some applications, the instrument is configured to incise the prostatic capsule.

There is additionally provided, in accordance with some applications of the present invention, apparatus for treating an obstruction of a prostatic urethra of a subject, including:

a balloon device having a balloon; and

at least one cutting element coupled to the balloon and configured to perform an action on a prostate capsule of the subject, the action being selected from the group consisting of: incising and excising.

For some applications, the at least one cutting element is movable with respect to the balloon.

There is further provided, in accordance with some applications of the present invention, a method for treating an obstruction of a prostatic urethra of a subject, including:

accessing a prostate of the subject with an access device;

introducing a balloon to a vicinity of the prostate via the access device, the balloon having cutting elements coupled thereto;

inflating the balloon to create a space near the prostate; and

performing an action on a capsule of the prostate using the cutting elements, the action being selected from the group consisting of: incising and excising.

For some applications, the method further includes imaging the balloon introduction, and the performing of the selected action, with an imaging device.

There is additionally provided, in accordance with some applications of the present invention, a single port access apparatus, including:

a lighting source; and

a camera;

the apparatus defining at least one working channel, and the apparatus being configured to treat an obstruction of a prostatic urethra of a subject.

For some applications, the apparatus further includes camera-lens cleaning equipment, and the apparatus defines a lumen configured such that the camera-lens cleaning equipment can be inserted through the lumen.

For some applications, the apparatus further includes a fluid, and the apparatus defines a lumen configured such that the fluid insufflates a space between a prostate and a pubis of the subject by being passed through the lumen.

There is further provided, in accordance with some applications of the present invention, a method for treating an obstruction of a prostatic urethra of a subject, including executing a procedure via only one access port, wherein executing the procedure includes:

accessing a prostate of the subject with an endoscope by inserting the endoscope via the access port, the endoscope having at least one working channel; and

performing an action on a prostate capsule of the prostate via the at least one working channel of the endoscope, the action being selected from the group consisting of: incising and excising.

For some applications, the method further includes creating a space in a vicinity of the prostate by inflating a balloon in the vicinity prior to performing the selected action.

There is additionally provided, in accordance with some applications of the present invention, apparatus for treating an obstruction of a prostatic urethra of a subject, including:

an access needle; and

a cutting instrument, configured to perform an action on a prostate capsule of the subject, via the access needle, the action being selected from the group consisting of: incising, and excising.

For some applications, the apparatus further includes a stylet configured to facilitate introduction of the needle into the subject.

There is further provided, in accordance with some applications of the present invention, a method for treating an obstruction of a prostatic urethra of a subject, including:

introducing at least one access needle from a lower abdomen of the subject toward an anterior surface of a prostate of the subject by following a retropubic approach; and

performing an action on a capsule of the prostate, via the at least one access needle, the action being selected from the group consisting of: incising, and excising.

For some applications, the method further includes imaging the performing of the selected action, using an imaging device selected from the group consisting of: an ultrasound imaging device, an endoscopic imaging device; an MRI imaging device; and a CT imaging device.

For some applications, the method further includes inserting a balloon to a vicinity of the anterior surface via the access needle, and creating a space in the vicinity by inflating the balloon.

There is further provided, in accordance with some applications of the present invention, apparatus for treating an obstruction of a prostatic urethra of a subject, including a flexible device configured to be placed inside a prostate of the subject and to reduce a constrictive force on a portion of a body of the subject, by exerting a radial force on the prostate, the portion being selected from the group consisting of: a urethra and a bladder neck.

For some applications, the device is configured to be implanted inside the subject's body.

There is further provided, in accordance with some applications of the present invention, a method for treating an obstruction of a prostatic urethra of a subject, including reducing a constrictive force on a portion of a body of the subject, by exerting a radial force on a prostate of the subject, by non-transurethrally placing a flexible device inside the prostate, the portion being selected from the group consisting of: a urethra and a bladder neck.

For some applications, non-transurethrally placing the flexible device inside the prostate includes introducing the device from a lower abdomen of the subject toward an anterior surface of the prostate by following a retropubic approach.

There is additionally provided, in accordance with some applications of the present invention, a method for treating an obstruction of a prostatic urethra of a subject, including creating a lesion in a prostate capsule of a prostate of the subject, the lesion being selected from the group consisting of: an incision and an excision, and preventing the lesion from closing by exerting a radial force on the prostate, by placing a flexible device inside the prostate, subsequent to creating the lesion.

There is additionally provided, in accordance with some applications of the present invention, a method for treating an obstruction of a prostatic urethra of a subject, including creating a lesion in a prostate capsule of the subject, the lesion being selected from the group consisting of: an incision and an excision, and preventing the lesion from closing by placing at least one flexible device in a vicinity of the lesion.

There is additionally provided, in accordance with some applications of the present invention, a method, including performing an action on tissue of a subject by transperineally accessing the tissue, the action being selected from the group consisting of: incising, and excising, the tissue being selected from the group consisting of: prostate capsule tissue and prostate tissue of the subject.

For some applications, performing the selected action on the tissue includes treating an obstruction of a prostatic urethra of the subject.

For some applications, the method further includes imaging the performing of the action using transrectal ultrasound imaging.

For some applications, performing an action selected from the group consisting of: accessing the tissue, excising the tissue, and incising the tissue, includes performing the selected action using an aid selected from the group consisting of: a computer-aid and a robotic-aid.

There is additionally provided, in accordance with some applications of the present invention, a method including performing an action on tissue of a subject by extra-urethrally accessing the tissue, the action being selected from the group consisting of: incising, and excising, the tissue being selected from the group consisting of: prostate capsule tissue and prostate tissue of the subject.

For some applications, accessing the tissue includes accessing the tissue without damaging the urethra.

For some applications, accessing the tissue includes accessing the tissue without puncturing the urethra.

For some applications, the method further includes imaging the performing of the selected action using transrectal ultrasound imaging.

For some applications, performing the selected action on the tissue includes using an incision technique selected from the group consisting of: laser incision, RF incision, plasma incision, ultrasound incision and electrical incision.

There is further provided, in accordance with some applications of the present invention, apparatus including a shaft having a sharp distal end, the apparatus being configured to facilitate an extraurethral action on the tissue by penetrating the tissue, the action being selected from the group consisting of: incising, and excising, the tissue being selected from the group consisting of: prostate capsule tissue and prostate tissue.

For some applications, the apparatus further includes a movable arm at a distal portion of the shaft.

For some applications, the arm is configured to press tissue located between the arm and the distal portion of the shaft, by the arm being at least partially closed with respect to the distal portion of the shaft.

For some applications, the apparatus is configured to apply a pressing force to the tissue and to perform coagulation and cutting of the tissue subsequently to applying the pressing force to the tissue.

For some applications, the apparatus includes a device configured to perform the selected action on the tissue, the device being selected from the group consisting of: a laser, an RF device, a plasma device, an ultrasound device, and an electrical device.

There is additionally provided, in accordance with some applications of the present invention, a method, including:

identifying a subject as suffering from a disorder selected from the group consisting of: benign prostatic hyperplasia and focal prostatic cancer; and

in response to the identifying:
percutaneously, transperineally inserting a resection tool into a prostate of the subject; and
using the resection tool, resecting a portion of the prostate, without contacting a urethra of the subject with the resection tool.

For some applications, the method further includes, in response to the identifying, percutaneously, transperineally performing an act selected from the group consisting of cutting a portion of a prostate capsule of the subject, and resecting a portion of a prostate capsule of the subject.

For some applications, performing the selected action includes perfoiming the selected action on the portion of the prostate capsule with the resection tool.

There is further provided, in accordance with some applications of the present invention, a method, including:

identifying a subject as suffering from a disorder selected from the group consisting of: benign prostatic hyperplasia and focal prostatic cancer; and

in response to the identifying:
percutaneously, transperineally inserting a tissue-collecting tool into a prostate of the subject; and
using the tissue-collecting tool, removing from the prostate, tissue of the prostate that has been resected, without contacting a urethra of the subject with the tissue-collecting tool.

There is further provided, in accordance with some applications of the present invention, a method, including:

identifying a subject as suffering from a disorder selected from the group consisting of: benign prostatic hyperplasia and focal prostatic cancer; and

in response to the identifying:
percutaneously, transperineally inserting a laser device into a prostate of the subject; and
using a laser beam generated by the laser device, ablating a portion of the prostate, without contacting a urethra of the subject with the laser device.

For some applications, the method further includes, in response to the identifying, percutaneously, transperineally performing an act selected from the group consisting of cutting a portion of a prostate capsule of the subject, and resecting a portion of a prostate capsule of the subject.

For some applications, performing the selected action includes performing the selected action on the portion of the prostate capsule using the laser beam.

There is further provided, in accordance with some applications of the present invention, apparatus for collecting, from inside a body of a subject, tissue of the subject, the apparatus including:

an elongate element configured to be inserted into the subject's body;

a tissue-collecting loop disposed at a distal end of the elongate element; and

a net disposed between the loop and the elongate element, and configured to collect the tissue inside the net by the loop being moved such that the net at least partially contains the tissue.

For some applications, the apparatus further includes a tissue resection device configured to:

percutaneously be inserted into an organ of the subject; and

resect tissue of the organ,

and the net is configured to collect the resected tissue of the organ.

There is further provided, in accordance with some applications of the present invention, a method for collecting, from inside a body of a subject, tissue of the subject, the method including:

inserting an elongate element into the subject's body, a tissue-collecting loop being disposed at a distal end of the elongate element, and a net being disposed between the loop and the elongate element; and

collecting the tissue inside the net by the loop being moved such that the net at least partially contains the tissue.

For some applications, the method further includes:

percutaneously inserting a resection tool into an organ of the subject; and

using the resection tool, resecting tissue of the organ,

and collecting the tissue inside the net includes collecting the resected tissue of the organ.

There is further provided, in accordance with some applications of the present invention, a method, including:

identifying a subject as suffering from benign prostatic hyperplasia; and

in response to the identifying:
percutaneously, transperineally inserting a resection tool to a vicinity of a prostate capsule of the subject; and
using the resection tool, resecting a portion of the prostate capsule, without contacting a urethra of the subject with the resection tool.

There is further provided, in accordance with some applications of the present invention, a method, including:

identifying a subject as suffering from benign prostatic hyperplasia; and

in response to the identifying:
percutaneously, transperineally inserting an incision tool to a vicinity of a prostate capsule of the subject; and
using the incision tool, incising a portion of the prostate capsule, without contacting a urethra of the subject with the incision tool.

There is further provided, in accordance with some applications of the present invention, a method, including:

identifying a subject as suffering from benign prostatic hyperplasia; and

in response to the identifying:
percutaneously, transperineally inserting a laser device to a vicinity of a prostate capsule of the subject; and
using a laser beam generated by the laser device, ablating a portion of the prostate capsule, without contacting a urethra of the subject with the laser device.

There is additionally provided, in accordance with some applications of the present invention, a method, including:

identifying a subject as suffering from a disorder of an organ of the subject; and

in response to the identifying:
inserting a shaft into the organ, not via a natural lumen of the subject;
using a laser beam emitted from a distal portion of the shaft, ablating a portion of the organ; and
cooling a vicinity of the portion of the organ by introducing a fluid to the vicinity, and, subsequently aspirating the fluid therefrom, the introduction and aspiration of the fluid being performed via the shaft, while the shaft is inside the organ.

For some applications, the organ includes an organ selected from the group consisting of: a prostate, a liver, and a kidney, and inserting the shaft into the organ includes inserting the shaft into the selected organ.

For some applications, inserting the shaft includes inserting a shaft having a diameter of less than 5 mm

For some applications, aspirating the fluid includes aspirating ablation products from the organ.

For some applications, ablating the portion includes ablating the portion without direct vision of the portion.

For some applications, the method further includes viewing the ablation.

For some applications, viewing the ablation includes using an imaging modality selected from the group consisting of: abdominal ultrasound imaging, transrectal ultrasound imaging, and transurethral ultrasound imaging.

For some applications, the organ includes the subject's prostate and identifying the subject includes identifying the subject as suffering from a disorder selected from the group consisting of: benign prostatic hyperplasia and focal prostatic cancer.

For some applications, the method further includes incising a prostate capsule of the subject.

For some applications, inserting the shaft includes inserting the shaft without damaging a urethra of the subject, and ablating the portion includes ablating the portion without damaging the urethra.

For some applications, inserting the shaft includes inserting the shaft transperineally into the subject's prostate.

There is further provided, in accordance with some applications of the present invention, apparatus for use with a cooling fluid, and for treating a subject suffering from a disorder of an organ, the apparatus including:

a laser device, including:
a shaft having a diameter of less than 5 mm,
a laser fiber disposed inside the shaft, and configured to ablate a portion of the selected organ by directing a laser beam toward the portion,
the shaft defining:
   an introduction lumen configured to facilitate cooling of a vicinity of the portion by introducing the cooling fluid to the vicinity, and
   an aspiration lumen configured to facilitate aspiration of the cooling fluid from the portion.

There is additionally provided in accordance with some applications of the present invention, a method, including:

identifying a subject as suffering from a disorder selected from the group consisting of: benign prostatic hyperplasia and focal prostatic cancer; and

in response to the identifying:
transperineally inserting a tool into a retropubic space of the subject; and
while at least a portion of the tool is disposed in the retropubic space, performing an action with respect to a portion of a prostate of the subject using the tool, the action being selected from the group consisting of: ablation, incision, imaging and resection.

For some applications, inserting the tool includes inserting the tool minimally-invasively.

For some applications, inserting the tool includes inserting the tool without damaging a urethra of the subject.

For some applications, performing the selected action includes ablating the subject's prostate using a laser.

For some applications, performing the selected action includes incising a prostate capsule of the subject's prostate.

For some applications, performing the selected action includes excising a portion of the subject's prostate.

For some applications,

the tool includes (a) an endoscope having a working channel, and (b) a treatment tool,

inserting the tool into the retropubic space includes (c) inserting the endoscope into the retropubic space and (d) inserting the treatment tool into the retropubic space via the working channel of the endoscope, and

performing the selected action includes (e) performing an action on the portion of the prostate, with the treatment tool, the action being selected from the group consisting of: ablation, incision and resection, and (f) imaging, with the endoscope, the action being performed by the treatment tool.

For some applications,

the tool includes (a) an endoscope and (b) a treatment tool,

inserting the tool into the retropubic space includes (c) inserting the endoscope into the retropubic space via a first access port in a perineum of the subject, and (d) inserting the treatment tool into the retropubic space via a second access port in the subject's perineum, and

performing the selected action includes (e) performing an action with the treatment tool selected from the group consisting of: ablation, incision and resection, and (f) imaging the action being performed by the treatment tool with the endoscope.

For some applications, the tool includes an endoscope, inserting the tool into the retropubic space includes inserting the endoscope into the retropubic space, and performing the selected action includes imaging the portion of the prostate with the endoscope.

For some applications, the method further includes transperineally inserting a treatment tool into the prostate, and, while the endoscope is imaging the portion of the prostate, performing an action on the portion of the prostate with the treatment tool, the action being selected from the group consisting of: ablation, incision, and resection.

For some applications, the method further includes enlarging the retropubic space prior to performing the selected action.

For some applications,

the tool includes (a) a balloon, and (b) a treatment tool,

inserting the tool into the retropubic space includes:
first, inserting the balloon into the retropubic space and enlarging the retropubic space by inflating the balloon, and
subsequently, inserting the treatment tool into the retropubic space, and

performing the selected action includes performing the selected action with the treatment tool.

For some applications,

the tool includes (a) a fluid-introduction device, and (b) a treatment tool,

inserting the tool into the retropubic space includes:
first, inserting the fluid-introduction device into the retropubic space and enlarging the retropubic space by introducing fluid directly into the retropubic space via the fluid-introduction device, and
subsequently, inserting the treatment tool into the retropubic space, and

performing the selected action includes performing the selected action with the treatment tool.

The present invention will be more fully understood from the following detailed description of embodiments thereof, taken together with the drawings, in which:

### BRIEF DESCRIPTION OF THE DRAWINGS

FIG. 1 is a schematic illustration of a tool for incising the capsule of the prostate, in accordance with some applications of the present invention;

FIG. 2 is a schematic illustration of the capsule of the prostate after incision, in accordance with some applications of the present invention;

FIG. 3A is a schematic illustration of a Y-shaped incision into the prostatic capsule, in accordance with some applications of the present invention;

FIG. 3B is a schematic illustration of the prostatic capsule after the Y-shaped incision, in accordance with another application of the present invention;

FIGS. 4-5 are schematic illustrations of an incision device inserted into the body of the subject, in accordance with respective applications of the present invention;

FIG. 6A is a schematic illustration of a balloon prior to inflation thereof, inserted into the body of the subject, in accordance with some applications of the present invention;

FIG. 6B is a schematic illustration of the balloon of FIG. 6A, now inflated, inserted into the body of the subject, in accordance with some applications of the present invention;

FIG. 6C is a schematic illustration of an incision device inserted into the body of a subject, after withdrawal of the balloon, in accordance with some applications of the present invention;

FIG. 7A is a schematic illustration of a balloon prior to the inflation thereof, inserted into the body of a subject in accordance with another application of the present invention;

FIG. 7B is a schematic illustration of the balloon of FIG. 7A, now inflated, inserted into the body of a subject, in accordance with some applications of the present invention;

FIG. 7C is a schematic illustration of a balloon, in accordance with an alternative application of the present invention;

FIGS. 8A-F are schematic illustrations of a balloon for inserting into a vicinity of the anterior side of the subject's prostate, in accordance with some applications of the present invention;

FIGS. 9A-C are schematic illustrations of a balloon which comprises cutting elements, in accordance with some applications of the present invention;

FIGS. 10A-C are schematic illustrations of a balloon that unfolds, in accordance with some applications of the present invention;

FIGS. 11A-D are cross-sectional schematic illustrations of balloon cutting elements, in accordance with respective applications of the present invention;

FIGS. 12A-D are cross-sectional schematic illustrations of dynamic cutting elements, in accordance with respective applications of the present invention;

FIG. 13 is a schematic illustration of a single port access endoscope, in accordance with some applications of the present invention;

FIGS. 14A-B are schematic illustrations of an access needle which serves as a working channel, in accordance with some applications of the present invention;

FIGS. 15A-B are schematic illustrations of a balloon, in accordance with some applications of the present invention;

FIG. 16 is schematic illustration of a cutting instrument, in accordance with some applications of the present invention;

FIGS. 17A-C are schematic illustrations of the tip of the cutting element shown in FIG. 16, in accordance with respective applications of the present invention;

FIGS. 18A-B are schematic illustrations of the introduction of an access needle, a balloon, and a cutting instrument to the anterior side of the subject's prostate, in accordance with some applications of the present invention;

FIGS. 19-20 are schematic illustrations of a flexible device designed to prevent closure of the cuts in the prostate capsule, in accordance with respective applications of the present invention;

FIGS. 21A-B are schematic illustrations of apparatus for cutting and/or coagulating soft tissue, in accordance with some applications of the present invention;

FIG. 22 is a schematic illustration of a plastic hinge used with the apparatus shown in FIGS. 21A-B, in accordance with some applications of the present invention;

FIGS. 23A-B are schematic illustrations of apparatus used for cutting and/or coagulating soft tissue, in accordance with an alternative application of the present invention;

FIGS. 24A-E are schematic illustrations of a procedure for performing prostate capsulotomy, in accordance with some applications of the present invention;

FIG. 25 is a schematic illustration of a laser device for cutting and/or coagulating soft tissue, in accordance with some applications of the present invention;

FIGS. 26A-B are schematic illustrations of respective views of a prostate, portions of which have been resected, in accordance with some applications of the present invention;

FIGS. 27A-C are schematic illustrations of a prostate resection device, in accordance with some applications of the present invention;

FIGS. 28A-D are schematic illustrations of a laser device, in accordance with some applications of the present invention;

FIGS. 29A-E are schematic illustrations of respective steps of a procedure that is performed, in accordance with some applications of the present invention; and

FIG. 30 is schematic illustration showing a lateral view (sagittal section) of a procedure, in accordance with some applications of the present invention.

### DETAILED DESCRIPTION OF EMBODIMENTS

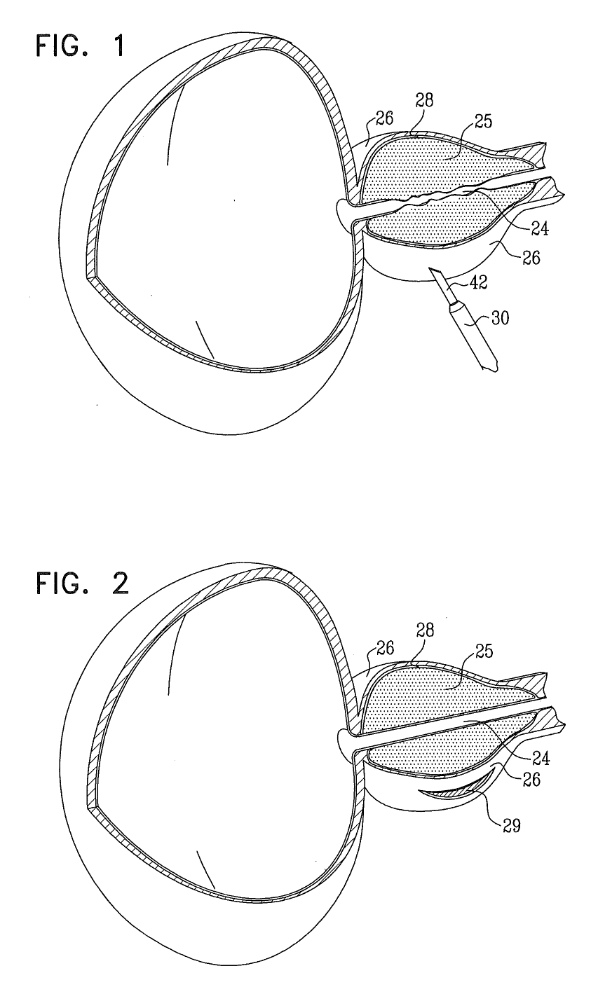

Reference is now made to FIG. 1, which is a schematic illustration of an incision device 42, which has been introduced via an introduction device 30, incising or excising at least a portion of a capsule 26 of a prostate 28 of a subject, in accordance with some applications of the present invention. A benign prostatic hyperplasia (BPH) 25 in prostate 28, or an obstruction, causes the constriction of a urethra 24 of the subject. Incision of prostatic capsule 26 by incision device 42 relieves pressure on the constricted portion of urethra 24, because it allows the expansion of prostate 28 in an outward direction.

Introduction device 30 typically comprises an endoscopic imaging device, an ultrasound device, a CT imaging device, an MRI imaging device, a cystourethroscope, or a laparoscope, in order to facilitate the passage of incision device 42 to prostatic capsule 26. The incision device typically comprises a cold knife, a cauterizing tool, a laser, or another cutting tool that is suitable for incising the prostatic capsule, for example, as described hereinbelow. For some applications, a multi-lumen endoscope, or an access needle is used for introduction device 30, as described hereinbelow. For some applications, the incision device is not inserted via an introduction device.

Reference is now made to FIG. 2, which is a schematic illustration of prostatic capsule 26 after an incision 29 has been made, in accordance with some applications of the present invention. Incision device 42 typically incises prostatic capsule 26 one or more times, for example, to create one to three or four to ten incisions. FIG. 2 shows prostatic capsule 26 after a single incision has been made, thereby relieving the urethral constriction, as shown.

For some applications, incision device 42 incises prostatic capsule 26 in a superior-inferior direction. In other applications, incision device 42 incises prostatic capsule 26 in a medial-lateral direction. In yet another application, incision device 42 incises prostatic capsule 26 multiple times. For some applications the incisions are in the same direction, and in other applications, the incisions are in different directions. Alternatively, incision device 42 excises at least a portion of prostatic capsule 26.

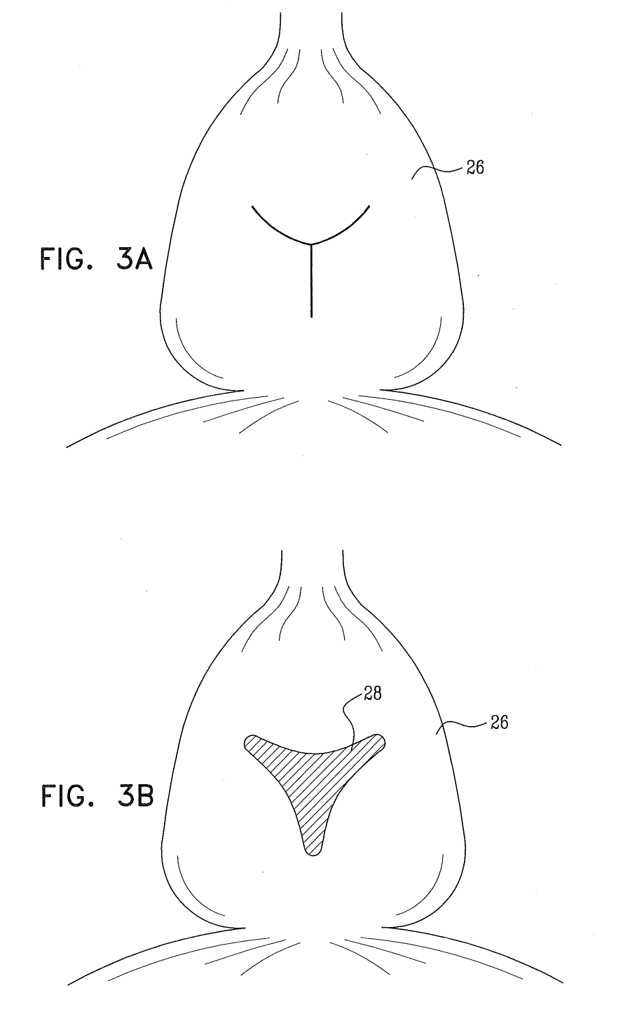

Reference is now made to FIG. 3A, which is a schematic illustration of prostatic capsule 26 with a Y-shaped incision, created to relieve pressure within prostate 28, in accordance with some applications of the present invention.

Reference is now made to FIG. 3B, which is a schematic illustration of prostatic capsule 26 following creation of the Y-shaped incision shown in FIG. 3A, in accordance with some applications of the present invention.

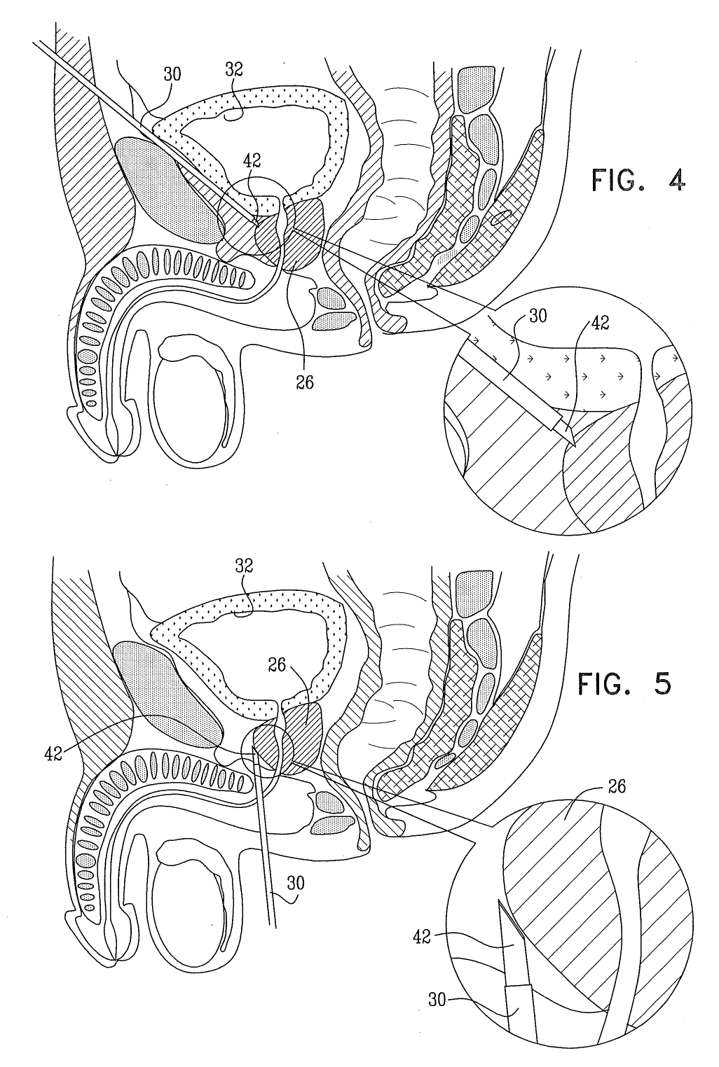

Reference is now made to FIGS. 4-5, which are schematic illustrations of incision device 42 inserted into the body of the subject, in accordance with respective applications of the present invention. FIG. 4 schematically illustrates transabdominal access by incision device 42 to prostatic capsule 26. FIG. 5 schematically illustrates transperineal access of incision device 42 to prostatic capsule 26. (It is noted that introduction device 30 is introduced such that the introduction device does not traverse the subject's urethra.) For some applications, incision device 42 is inserted toward prostatic capsule 26 transurethrally.

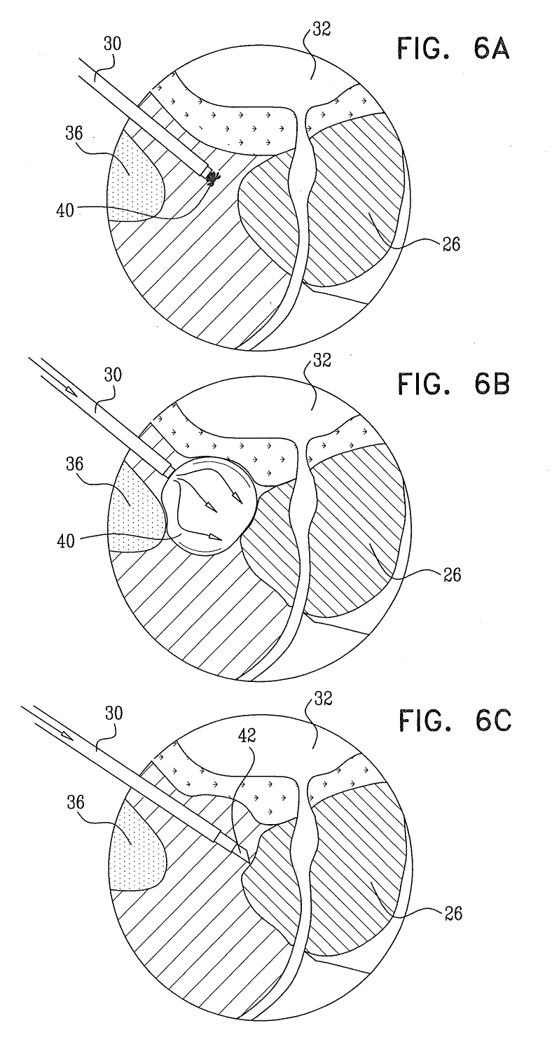

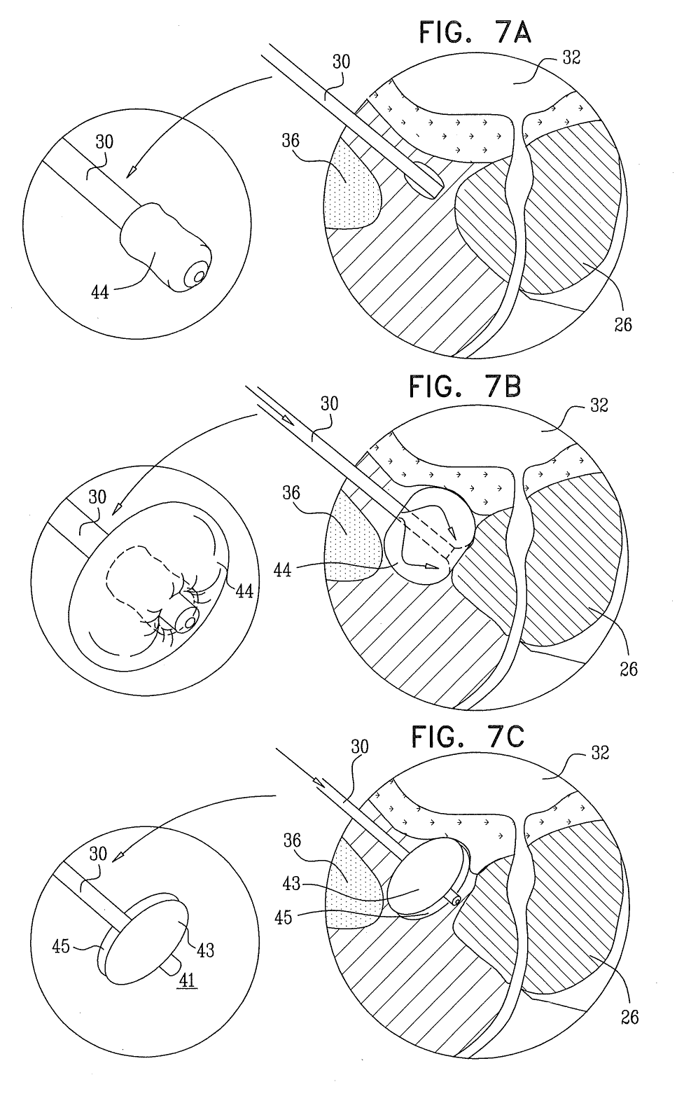

Reference is now made to FIG. 6A, which is a schematic illustration of a balloon 40 prior to inflation thereof, inserted into the body of a subject, in accordance with some applications of the present invention. Balloon 40 is typically advanced transabdominally to prostate 26, via introduction device 30. For some applications, the balloon passes around pubic bone 36, i.e., a retropubic approach. Alternatively, the balloon is inserted transperineally, or via a different approach. For some applications, the balloon is not inserted via an introduction device.

Reference is now made to FIG. 6B, which is a schematic illustration of balloon 40 inflated, in accordance with some applications of the present invention. On inflation, balloon 40 pushes prostate 28, surrounded by prostatic capsule 26, in a posterior direction, and/or pushes a bladder 32 of the subject in a superior direction, creating a space to facilitate capsulotomy. Although balloon 44, when maximally inflated, is shown to have a generally spherical shape, the scope of the present invention includes having a differently-shaped balloon, such as a balloon having a shape as described herein.

Reference is now made to FIG. 6C, which is a schematic illustration of incision device 42 incising capsule 26 after deflation of balloon 40, in accordance with another application of the present invention. After deflation of the balloon, the space created by inflating balloon 40 is insufflated with air or carbon dioxide, providing and maintaining additional space for incision device 42 to perform the capsulotomy. While the space is maintained (typically by ongoing application of air or CO2), incision device 42 is used to make one or more incisions into prostatic capsule 26.

Reference is now made to FIG. 7A, which is a schematic illustration of a balloon 44 prior to inflation thereof, inserted into the body of a subject, in accordance with another application of the present invention. Balloon 44 surrounds the distal end of introduction device 30 (e.g., an endoscope, an access needle, or another suitable introduction device) and is advanced transabdominally to prostate 26 during advancement of introduction device 30. Alternatively, the balloon is inserted transperineally, or via a different approach.

Reference is now made to FIG. 7B, which is a schematic illustration of balloon 44 inflated, in accordance with some applications of the present invention. On inflation, balloon 44 pushes bladder 32 in a superior direction and/or pushes prostate 28 surrounded by capsule 26 in a posterior direction (as shown in FIG. 7B), creating a space to facilitate capsulotomy by incision tool 42. Although balloon 44 is shown having a generally spherical shape, when maximally inflated, the scope of the present invention includes having a different shaped balloon, such as balloons having shapes as described herein. Subsequently, the balloon is deflated and the prostate capsule is incised, in accordance with the techniques described with reference to FIG. 6C.

Reference is now made to FIG. 7C, which is a schematic illustration of two balloons 43 and 45 disposed on opposing sides of introduction device 30, for example, a left-balloon 43 and a right-balloon 45, in accordance with an alternative application of the present invention. For some applications, the left and right balloons are inflated on the left and right sides of the distal end of introduction device 30. While the balloons are inflated, one or more working spaces 41 are created in the vicinity of the distal end, and the prostate and/or prostate capsule is incised and/or excised using an instrument that is inserted into the working space, using techniques described herein. For some applications the working space is created distal to the balloons and to the introduction device. FIG. 7C includes a sagittal view of balloons 43 and 45 inserted in the subject's body, therefore a greater portion of left-balloon 43 is seen, than of right balloon 45. From an anterior or posterior perspective, one would observe the left and right balloons, respectively to the left and right of the distal end of the introduction device.

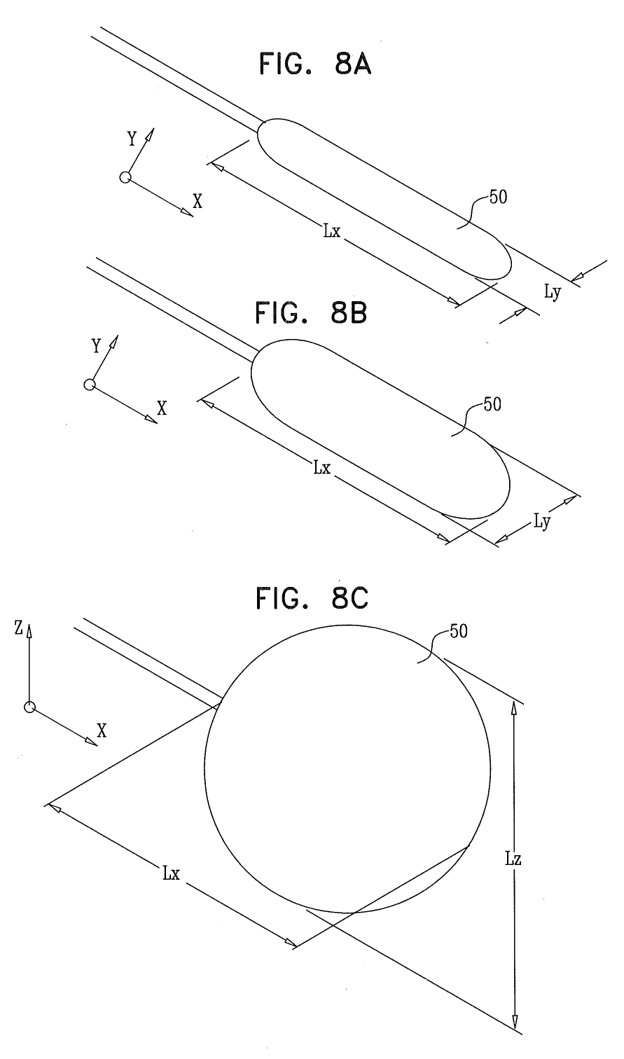

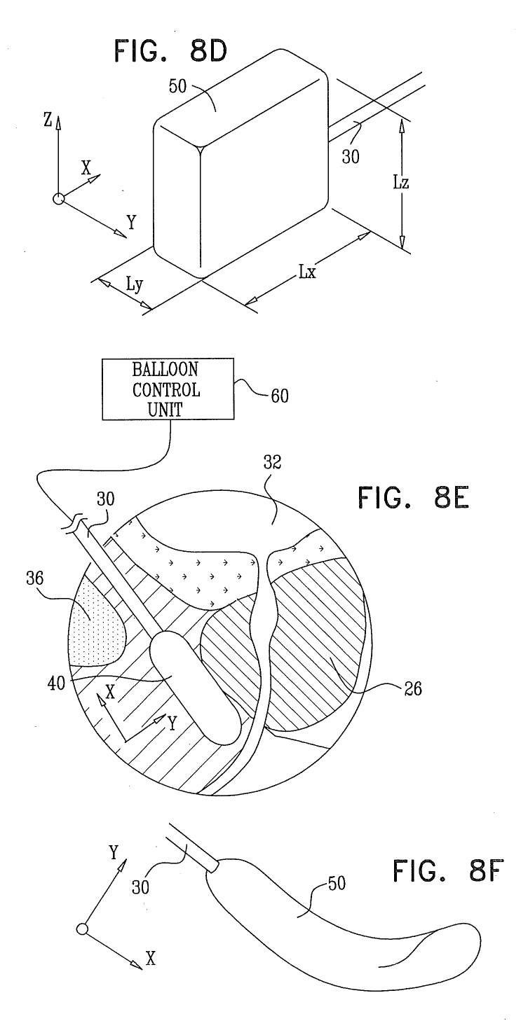

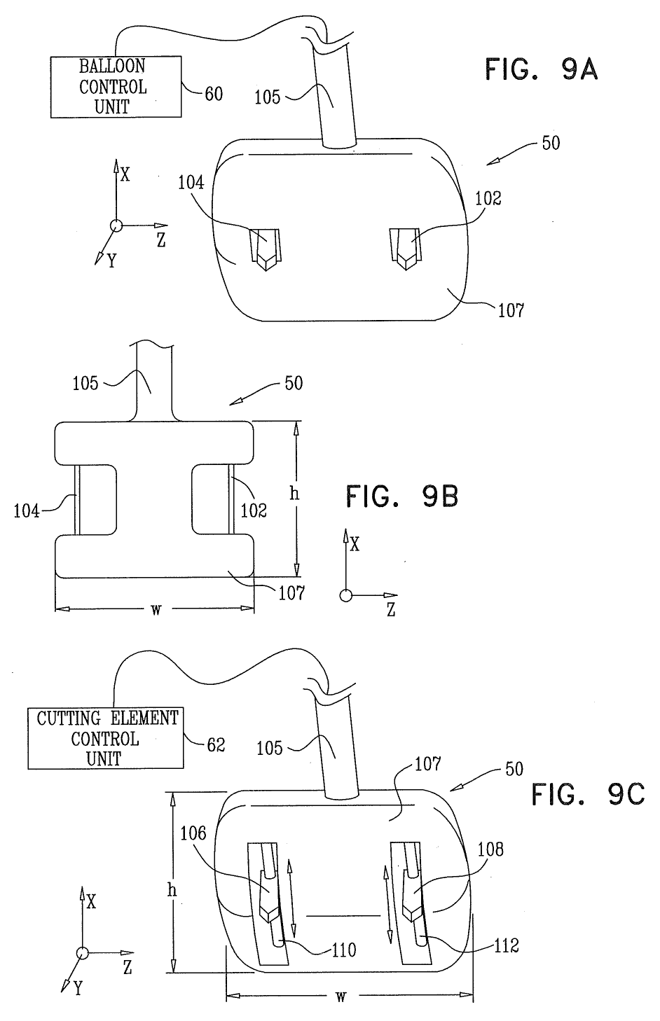

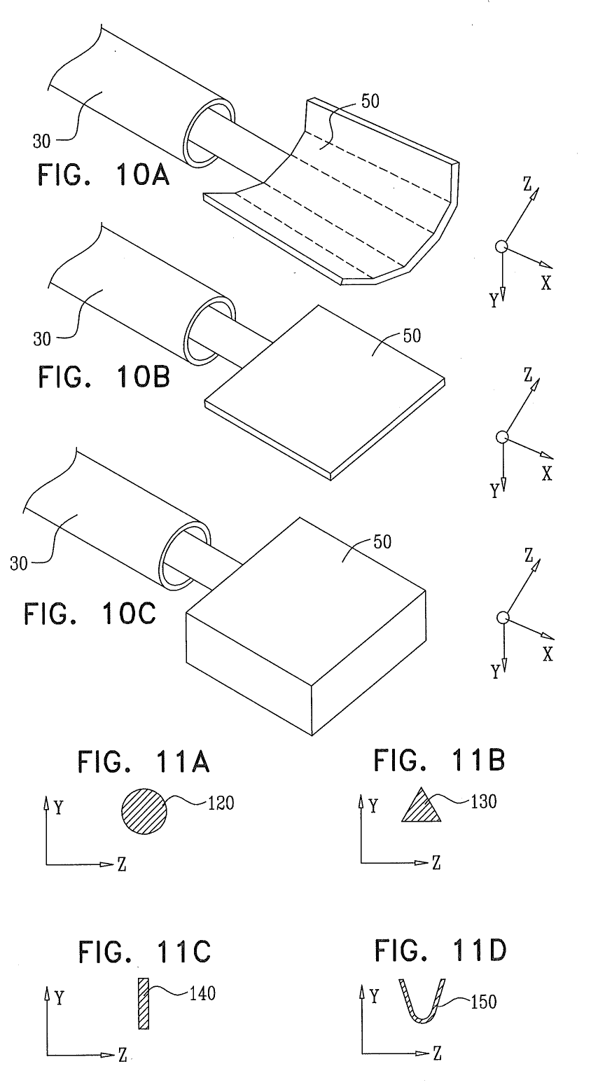

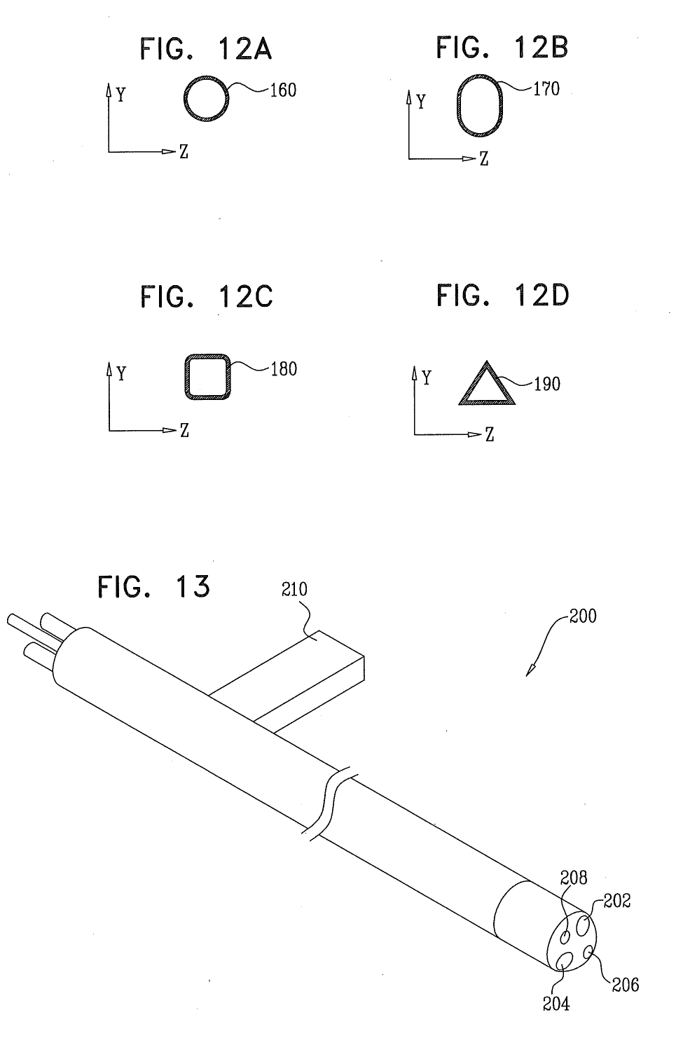

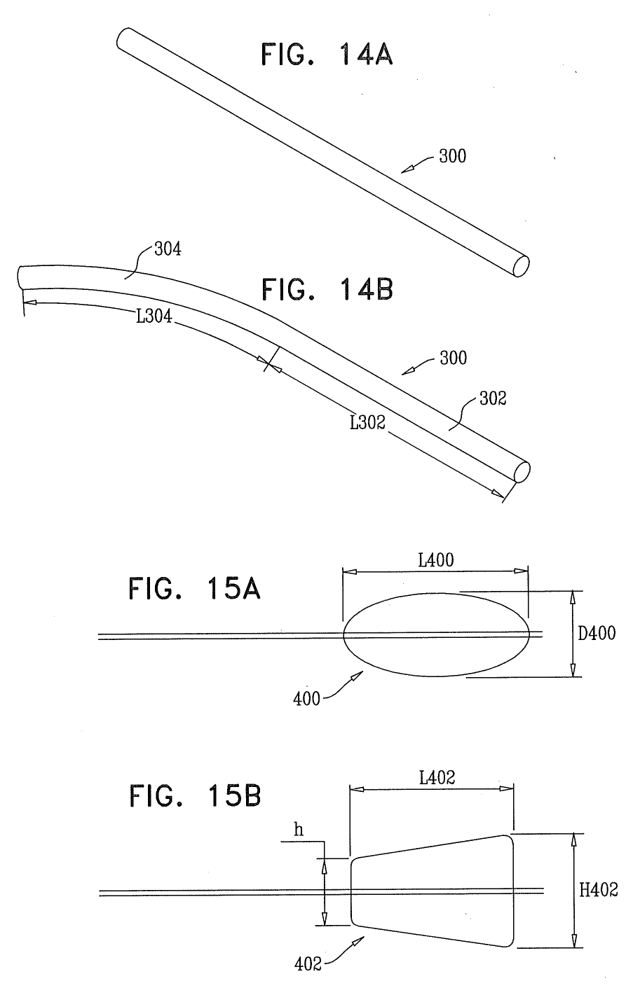

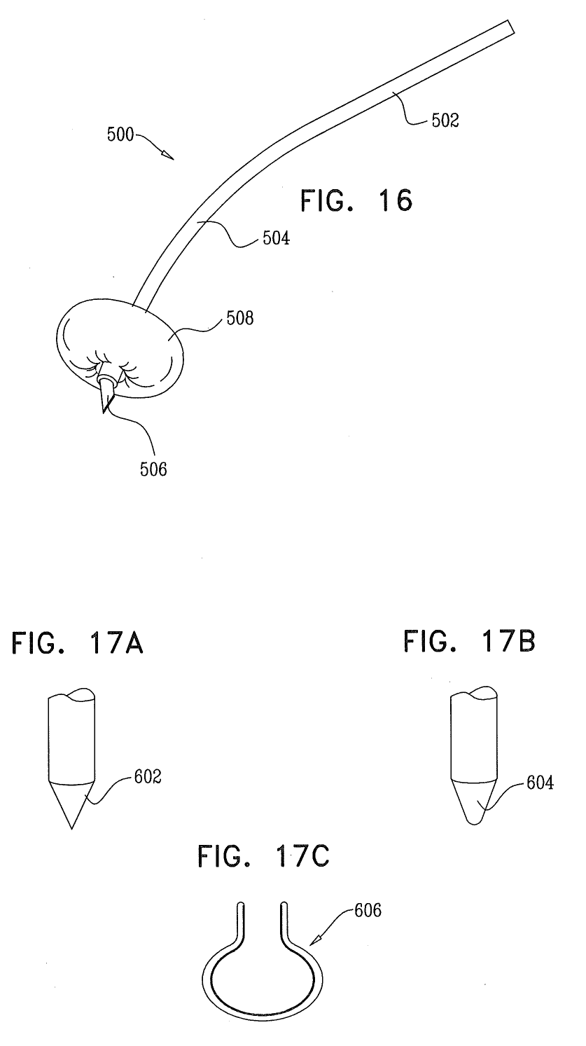

Reference is now made to FIGS. 8A-F, which are schematic illustrations of a balloon 50 for inserting into the vicinity of the anterior side of the subject's prostate, in accordance with some applications of the present invention. For some applications, balloon 50 is inflated so as create a space to facilitate capsulotomy by incision tool 42, using generally similar techniques to those described with reference to FIGS. 6A-C. Alternatively or additionally, cutting edges that are coupled to the balloon cut the prostate capsule while the balloon is in an inflated state, for example, as described with reference to FIGS. 9A-C.

The balloon is typically inserted into the vicinity of the anterior side of the subject's prostate, via introduction device 30, using an insertion technique described herein. The balloon is inflated by balloon control unit 60, such that the balloon creates a space in the vicinity of the anterior side of the prostate. Typically when the balloon is maximally inflated in the x and z directions the balloon has maximal lengths Lx and Lz, in each of the x and z directions, of less than 60 mm, and a maximal length Ly of 30 mm to 100 mm in the y direction, the x, y, and z directions being mutually perpendicular to each other.

FIG. 8A shows balloon 50 in an uninflated state, and FIG. 8B shows the balloon in a maximally-inflated state. For some applications, during inflation of the balloon, the balloon expands predominantly in the y direction. For example, the expansion of the balloon in the y direction (as measured by dividing maximal length Ly when the balloon is maximally inflated by maximal length Ly when the balloon is uninflated) is typically more than 200%, or more than 400% (e.g., 200% to 500%) of the expansion of the balloon in each of the x and z directions (expansion in the x and z directions being measured similarly). For some applications, using a balloon that expands predominantly in the y direction facilitates accurate placement of cutting elements on the balloon, with respect to the balloon, in the x-z plane. Since the balloon expands predominantly in the y direction, the position with respect to the balloon in the x-z plane, of the cutting elements which are fixed to the balloon, does not vary by a substantial amount. In this manner, the cutting elements are advanced in the y direction upon inflation of the balloon. The cutting elements having substantially fixed positions with respect to the balloon in the x-z plane allows the cutting elements to be aimed at and moved toward target tissue.

For some applications, balloon 50 has a round shape in the x-z plane, as shown in FIG. 8C. Alternatively the balloon has a rectangular shape in the x-z plane, as shown in FIG. 8D, or the balloon has a different shape in the x-z plane.

For some applications, the balloon is inserted into the vicinity of the anterior side of prostate 26 via an elongate element, for example, introduction device 30. For example, the balloon may be inserted via the abdomen (as shown in FIG. 8E), or transperineally, using the techniques described herein. When the balloon is in the vicinity of the prostate, the balloon is inflated (typically, by balloon control unit 60), such that it creates space in the vicinity of the anterior side of the prostate.

FIG. 8F is a schematic illustration of a cross section of balloon 50 in the x-y plane while the balloon is in an inflated state, in accordance with some applications of the present invention. For some applications, the balloon is curved, as shown, to facilitate contact between the surface of the balloon and the anterior surface of the prostate. Typically, the balloon is configured to be curved upon being inflated by shaping the balloon in a mold, using techniques that are known in the art. For some applications, techniques that are used with The Cutting Balloon® manufactured by InterVentional Technologies Inc. (CA, USA) are practiced in combination with balloon 50.

Reference is now made to FIGS. 9A-B, which are schematic illustrations of balloon 50, or a different balloon, comprising one or more cutting elements 102 and/or 104, for example, cutting wires, or cutting blades, in accordance with respective applications of the present invention. The cutting elements incise or excise the prostate capsule and, for some applications, the cutting elements additionally incise or excise prostate tissue. Although FIGS. 9A-B show specific numbers and orientations of cutting elements, the scope of the present invention includes using different numbers of cutting elements and cutting elements that are oriented differently. For some applications, balloon 50 is introduced, transabdominally (for example, retropubically), towards the prostate of a patient, for example, via introduction device 30. Alternatively, the balloon is inserted transperineally. When the balloon is positioned in a vicinity of the anterior side of the subject's prostate, the balloon is filled with a compressible or non-compressible fluid, such as air, carbon dioxide or water, via tube 105. For some applications, balloon control unit 60 controls the inflation of the balloon. Typically, the inflation of the balloon creates space on the anterior side of the prostate, for example, between the anterior surface of the prostate and the posterior surface of the pubic bone. Following balloon inflation, balloon 50 is typically substantially in contact with the anterior surface of the prostate. Cutting element 102 and/or 104, which is coupled to a surface 107 of the balloon, is used to incise and/or excise the prostate capsule using techniques that are known in the art, and/or techniques as described herein. For some applications, the cutting element protrudes from surface 107 of the balloon. Alternatively, the cutting element is disposed beneath surface 107. For some applications, the cutting element is substantially immobile with respect to surface 107, in the x-z plane. In such applications, the prostate capsule is typically cut by moving the balloon with respect to the prostate capsule. For some applications, one or more of the following cutting techniques or instruments are used to incise and/or excise the prostate capsule: a knife, a sharp edge, diathermia, a harmonic scalpel (for example, the Harmonic Scalpel® manufactured by Johnson and Johnson), a monopolar or bipolar device (e.g., a LigaSure™ device manufactured by Valleylab), or laser, such as green laser.

Reference is now made to FIG. 9C, which is a schematic illustration of balloon 50, or a different balloon, including one or more dynamic cutting elements 106 and/or 108 mounted on a wire(s) 110, 112 or similar construction(s), in accordance with some applications of the present invention. The dynamic cutting elements are substantially mobile with respect to surface 107, in the x-z plane. In such applications, the prostate capsule is cut by moving the cutting elements with respect to surface 107, in the x-z plane, typically while surface 107 remains substantially stationary. The wires typically facilitate movement of the cutting elements during the incision and/or excision of the prostate capsule. For some applications, a cutting element control unit 62 controls the motion of the cutting elements.

For some applications, insertion of balloon 50, as shown in FIGS. 9A-C, is guided by an imaging device, for example, an endoscopic, ultrasound (e.g., transrectal ultrasound), CT and/or MRI imaging device. For some applications, balloon 50 and the imaging device are introduced into the subject's body via a single access port, for example, access needle 300 described hereinbelow.

Reference is now made to FIGS. 10A-C, which are schematic illustrations of balloon 50, in accordance with some applications of the present invention. For some applications, balloon 50 is in a folded disposition during insertion of the balloon into the subject's body. When the balloon emerges from the distal end of the insertion tool (for example, introduction device 30), the balloon is inflated. Inflation of the balloon initially, predominantly results in the unfolding of the balloon in the x-z plane (FIG. 10B). Subsequently, inflation of the balloon expands the balloon, for example, predominantly in the y direction (FIG. 10C), as described hereinabove. For some applications, using a folded balloon facilitates accurate placement of cutting elements on the balloon with respect to the balloon, in the x-z plane. For example, each of the cutting elements is disposed on a respective fold of the folded balloon. The placement of a cutting element on a given fold will result in the element having a specific position with respect to the balloon in the x-z plane, subsequent to the unfolding of the balloon. The element having a specific position with respect to the balloon in the x-z plane allows the cutting element to be aimed at and moved toward a target.

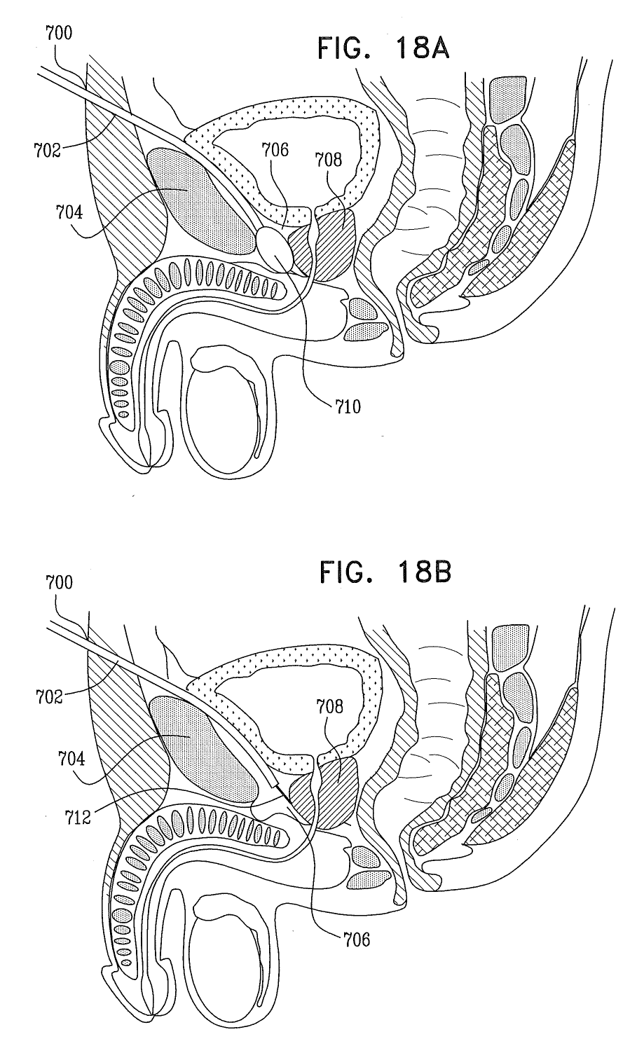

Reference is now made to FIGS. 11A-11D, which are schematic cross-sectional illustrations of cutting elements 102 and/or 104 shown in FIGS. 9A-B, in accordance with respective applications of the present invention. In respective applications, one or more of the cutting elements has:

a round cross section 120 (FIG. 11A);

a triangle cross section 130 (FIG. 11B), configured, for example, such that a sharp cutting edge faces the prostate;

a rectangular cross section 140 (FIG. 11C), that typically allows a surgeon to create a cut, the width and depth of the cut being in accordance with the width and depth of cutting element 140; and/or

a generally semi-circular cross section 150 (FIG. 11D), that typically allows a surgeon to create a relative wide cut, the cut edges being substantially spaced from each other. Typically, this reduces the risk of cut closing. The semi-circle is oriented such that the closed portion of the semi-circle faces the prostate.

Reference is now made to FIGS. 12A-D, which are schematic cross-sectional illustrations of dynamic cutting elements 106 and/or 108, shown in FIG. 9C, in accordance with respective applications of the present invention. Wires 110 and 112, shown in FIG. 9C, are typically shaped in accordance with the cross section of the cutting elements. In respective applications, one or more of the dynamic cutting elements has:

a round cross section 160 (FIG. 12A);

an oval shape cross section 170 (FIG. 12B);

a square cross section 180 (FIG. 12C); and/or

a triangle cross section 190 (FIG. 12D).

Reference is made now to FIG. 13, which is a schematic illustration of a single port endoscope 200, which, typically, allows a surgeon to perform an entire procedure as described herein, using a single access port, in accordance with some applications of the present invention. The endoscope comprises several lumens, for example, a lumen 202 for a camera and dedicated light source, a working channel 204, through which prostate capsule and optionally prostate tissue are cut using a soft-tissue cutting instrument, and an irrigation/suction lumen 206 for camera lens cleaning. For some applications, the device comprises an additional working lumen 208, for example, for the insertion of gas for insufflating the space near the prostate. Device 200 comprises a handle 210 at its proximal section. The endoscope shaft typically has a diameter of 6 mm to 8 mm.

Reference is made now to FIGS. 14A-B, which are schematic illustrations of an access needle 300 that serves as a working channel, in accordance with respective applications of the present invention. For some applications, access needle 300 is used as introduction device 30, described herein. In FIG. 14A, a straight needle 300 is shown. For some applications, needle 300 is constructed from a flexible material (e.g., nitinol, and/or a polymeric material) to allow its introduction via a straight and/or a curved path within the patient's body. FIG. 14B illustrates needle 300, in accordance with an alternative application. The needle shown in FIG. 14B has a proximal straight section 302 and a curved distal section 304 that is designed to comply with the lower abdomen anatomy, for example. Typically, a length L302 of the straight portion is 30 mm to 100 mm, and a length L304 of the straight portion is 20 mm to 100 mm. Introduction of access needle 300 may be facilitated by the use of a stylet (not shown). For example, a flexible and blunt tip stylet is inserted and, subsequently, the needle is passed over the stylet during needle introduction. Following the insertion of the needle, the stylet is removed.

Reference is now made to FIGS. 15A-B, which are schematic illustrations of a balloon device 400 or 402, that is introduced via needle 300, to create a void near the prostate, for example between the anterior surface of the prostate and the posterior surface of the pubic bone, in accordance with respective applications of the present invention. The balloon may have, for example, a round or elliptical shape 400 (FIG. 15A) or a trapezoid shape 402 (FIG. 15B). For some applications, the dimensions of balloon 400, and/or balloon 402 (and/or balloon 50, described hereinabove), are selected in accordance with the anatomy of the subject. For some applications, a diameter D400 of elliptical balloon 400 when inflated is 8 mm to 20 mm, and a length of the balloon L400 is less than 60 mm. For some applications, a height H402 of the larger parallel side of a trapezoidal balloon when inflated is 8 mm to 20 mm, a height h of the smaller parallel side of the trapezoidal balloon is 3 mm to 15 mm, and a length of the balloon L402 is less than 60 mm. For some applications, balloon 50 described hereinabove is inserted via access needle 300. During a procedure, balloon 400 is filled with a compressible fluid, a non-compressible fluid, and/or an ultrasound opaque substance in order to create a working space in a vicinity of the prostate. Following expansion of balloon 400, balloon 400 is deflated and removed. For some applications, an incision device cuts the prostate capsule, while the balloon is inflated. Alternatively, the balloon is deflated (and, optionally, the space in which the balloon was disposed is insufflated with a fluid) and, subsequently, an incision device cuts the prostate capsule.

Reference is now made to FIG. 16, which is a schematic illustration of a cutting instrument 500 that is introduced via access needle 300, or via a different introduction device, in order to cut the prostate capsule, in accordance with some applications of the invention. Cutting instrument 500 comprises a proximal straight section 502 and a curved distal section 504. For some applications, the lengths of the straight and curved sections comply with the lengths of the straight and curved sections of access needle 300. Typically, the shape of the device facilitates introduction of the device by complying with the lower abdomen anatomy, for example. A cutting element 506 is disposed at the distal end of the instrument. For some applications, the cutting element is disposed at an angle with respect to the portion of the device immediately proximal to the cutting element (as shown). For some applications, the disposition of the cutting element facilitates the cutting function. For some applications, a limiting structure 508 at the distal end of the device, for example, a balloon (as shown), or a pulley, limits the penetration into tissue of cutting element 506 to a pre-defined depth.

In accordance with some applications of the invention, cutting instrument 500 comprises a flexible stylet with a cutting element at its distal tip. Reference is now made to FIGS. 17A-C, which are schematic illustrations of a stylet cutting tip, in accordance with respective applications of the invention. In accordance with respective applications, the stylet has:

a conical tip 602 (FIG. 17A);

a blunt cutting tip 604 (FIG. 17B); and/or

a curved edge 606 (FIG. 17C) that typically removes a wide section of soft tissue. For some applications, other cutting tips and/or cutting techniques (e.g., diathermia and green laser) are used.

In accordance with some applications of the present invention, an imaging device, such as an endoscope, an ultrasound probe (e.g., a transrectal ultrasound probe), and/or a CT or MRI imaging device, is used in combination with needle 300 shown in FIGS. 14A-B, to guide needle 300 and to facilitate incision of the prostate capsule.

Reference is now made to FIGS. 18A-B, which are schematic illustrations of respective steps of a surgical procedure, in accordance with some applications of the present invention. FIG. 18A shows an entry point 700 of an access needle 702 above the pubic bone 704. For some applications, access needle 702 is generally similar to access needle 300 described hereinabove. For some applications, access needle includes a flexible stylet (not shown). Needle 702 follows the pubis until the needle reaches the retropubic space 706, anterior to the prostate 708 (i.e., a retropubic approach). Following positioning of needle 702, the stylet is removed and, optionally, a balloon device 710 is introduced and inflated. For some applications, balloon 710 is generally similar to balloon 50, 400, and/or 402 described hereinabove. FIG. 18B shows the introduction of a cutting instrument 712 via needle 702. Cutting instrument 712, which is typically a cutting instrument as described herein, creates one or more incisions and/or excisions at designated locations in the prostate capsule (with or without incising or excising prostate tissue). For some applications, the procedure is guided by an imaging device, as described hereinabove.

Reference is now made to FIG. 19, which is a schematic illustration of apparatus 800 that is used following the creation of one or more cuts 801 and/or 802 by incision and/or excision of the prostate capsule 804, in accordance with some applications of the present invention. For some applications, apparatus 800 prevents the cuts from re-closing. FIG. 19 is an inferior-superior view, thus only the ends of the relatively anterior cuts 801, 802 are shown in FIG. 19. Urethra 803 is also shown. For some applications, a flexible, generally circular, oval, or (as shown) omega-shaped implant 800 is placed around at least a portion of the circumference of the prostate. Ends 806 and 808 of the element are anchored in the prostate such that cuts 801, 802 are enclosed by the element. Device 800 is configured such that an outward radial force F forces the device toward an open configuration. For example, the device may comprise a shape-memory alloy, such as nitinol. Force F typically prevents closure of the cuts. For some applications, device 800 is non-transurethrally implanted in the subject's prostate without the prostate being incised (apart from incisions that are necessary for the implantation of device 800). The device exerts an outward radial force, as described, thus reducing the constrictive force on the prostatic urethra. For some applications, the device is implanted via a transabdominal, e.g., a retropubic, approach.

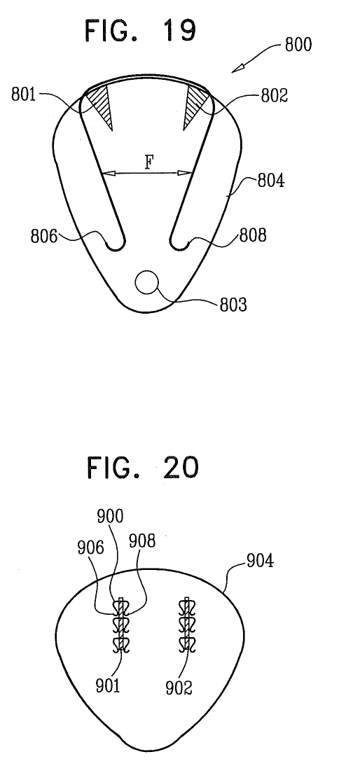

Reference is now made to FIG. 20, which is a schematic illustration of apparatus 900, which is used following the production of one or more cuts 901 and/or 902 by incision and/or excision of the prostate capsule 904, in accordance with some applications of the present invention. FIG. 20 is an anterior view of the prostate. For some applications, apparatus 900 prevents the cuts from re-closing. For some applications, at least one flexible, generally circular, oval, or (as shown) omega-shaped implant 900, is placed in a vicinity of each cut 901 and/or 902, so that its ends 906, 908 are anchored in the prostate. Implant 900 prevents the cuts from reclosing in a generally similar manner to omega-shaped implant 800 described with reference to FIG. 19.

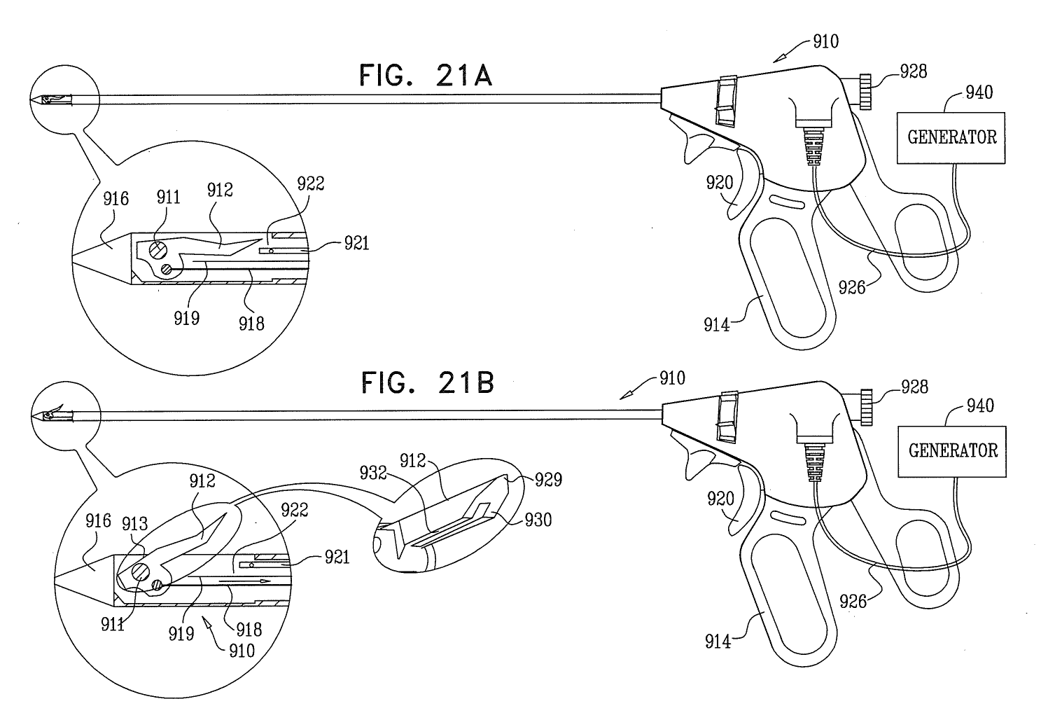

Reference now is made to FIGS. 21A-B, which are schematic illustrations of a device 910 intended for cutting and/or coagulating soft tissue (“a cutting device”), such as prostate capsule and/or prostate tissue, in accordance with some applications of the present invention. Cutting device 910 includes a joint 911 at a distal portion of the device. An arm 912 articulates with shaft 922 of the cutting device at the joint, at a distal end 913 of the arm, as shown in FIG. 21B. For example, joint 911 may include a pin, as shown. In a closed position of the arm with respect to the shaft, a portion of the arm faces a portion of the shaft. Shaft 922 typically has a diameter of 3 mm to 6 mm, e.g., 4 mm to 5 mm

Arm 912 typically includes a penetration element 929 at a proximal portion thereof, as shown in FIG. 21B. For some applications, penetration element includes a sharp edge, as shown. Device 910 typically has a handle 914 at its proximal end. For some applications, arm 912 also includes one or more protrusions 930, as shown in FIG. 21B. Typically, an electrode 919 and/or a cutting element 921 are disposed on the shaft. For example, the cutting element may be a blade, and/or other cutting elements as described herein, or as are known in the art. For some applications, the cutting element is slidable coupled to the shaft. For some applications, one or more of protrusions 930 on arm 912 include a cutting element (e.g., a blade) 932, and/or monopolar or bipolar electrodes.

Optionally, the distal tip 916 of device 910 is pointed to facilitate introduction of device 910 through soft tissue. Alternatively, distal tip 916 may be blunt (not shown in FIG. 21A), and device 910 is introduced, for example, via a needle (such as access needle 300 described hereinabove) following the introduction of a trocar and/or stylet. Typically, device 910 is advanced toward the prostate using techniques described herein, for example, transabdominally or transperineally.

For some applications, arm 912 is connected to a connection element 918, for example, a cable, a rod, or a tube, which is connected at its proximal end to an activating mechanism, such as a button 920. During introduction of device 910 toward the prostate, arm 912 is in a closed configuration, within the device shaft 922, to prevent damage to the tissue, as shown in FIG. 21A. Following introduction and positioning of device 910, button 920 is pressed and arm 912 protrudes from a slot or a lumen at the distal end of device shaft 922 into an open configuration (FIG. 21B). The opening of the arm facilitates the penetration (for example, by penetration element 929) and, optionally, the mechanical incision of tissue, for example, tissue of the prostatic capsule. For some applications, following the penetration (through, for example, tissue of the prostatic capsule), the tissue to be cut is positioned in the space between open arm 912 and device shaft 922. When the tissue is positioned in the space the arm is at least partially closed. In applications according to which cutting elements 932 are blades, the tissue is cut by closing the arm.

For some applications, device 910 coagulates tissue (using, for example, RF energy, plasma and/or laser technology), to reduce tissue bleeding due to the cut. Typically, the tissue is coagulated prior to cutting the tissue, although, for some applications, the tissue is first cut and then coagulated. Typically, an electrode 919 is disposed within shaft 922. When arm 912 is closed, the tissue between the aim and the shaft is grasped and is pressed against the electrode and coagulated, by the electrode driving a current into the tissue. Device 910 is connected (typically via a cable 926) to a generator 940, for example, a bipolar or a monopolar generator. For some applications, electrode 919 is a monopolar electrode and is connected to a monopolar generator. For some applications, when a monopolar generator is used, a ground (return) plate is placed against the patient (e.g., between a bed on which the patient lies and the patient's leg) (not shown). For some applications, connection element 918 is an electrical conductor that is electrically connected to arm 912, and that is isolated from device shaft 922. Arm 912 and electrode 919 act as bipolar electrodes. Activation of the coagulation function may be achieved by, for example, pressing a button 928 or a foot pedal (not shown).

For some applications, electrode 919, and, optionally, connection element 918, are aluminum electrodes. For some applications, one or more of the electrodes are coated using two processes: the alodine process, and hard anodization. For some applications, coating the electrodes in the aforementioned manner results in the electrodes having a coating that is less than 100 microns, for example, less than 60 microns thick. For some applications, the electrodes are coated in a polymer (e.g., epoxy, and/or polyester), using techniques that are known in the art.

For some applications, the prostate capsule is cut in accordance with the following procedure: The distal portion of shaft 922 is positioned adjacent to a first cutting site of the prostate capsule, while arm 912 is in a closed position. The arm is opened and the capsule is penetrated by penetration element 929. Tissue of the capsule is then grasped between the arm and shaft 922. The arm is partially closed in order to coagulate the tissue, as described hereinabove. Subsequently to the coagulation of the tissue, a cutting element (e.g., blade 921) is advanced distally, (for example, by sliding the blade distally with respect to the shaft) thereby cutting the coagulated tissue. The aim is typically in a closed position during the cutting of the tissue and the blade advances into the slot in arm 912 between protrusions 930. For some applications, the grasped tissue is cut by blade 921 without the tissue first being coagulated. For some applications, a blade disposed in a different portion, for example, on protrusions 930 of arm 912 cuts the grasped tissue. For some applications, a different cutting element, as described herein, is used to cut the grasped tissue. Subsequently to the tissue being cut, the distal portion of shaft 922 is moved adjacent to a more proximal cutting site. The cutting (and/or the coagulation procedure) is repeated at the more proximal cutting site. The device cuts tissue of the prostate capsule sequentially at successively more proximal cutting sites by the distal portion of shaft 922 being moved to successively more proximal cutting sites, and the cutting (and/or the coagulation procedure) being repeated.

In accordance with respective applications, device 910 is inserted transperineally or transabdominally. When inserted transperineally, the device cuts tissue of the prostate capsule from a position that is either inside or outside the prostate. Typically, when the device is inserted transabdominally, the device cuts the prostate capsule from a position that is outside the prostate.

For some applications, shaft 922 is shaped in a similar shape to the shape of access needle 300, described with reference to FIGS. 14A-B.

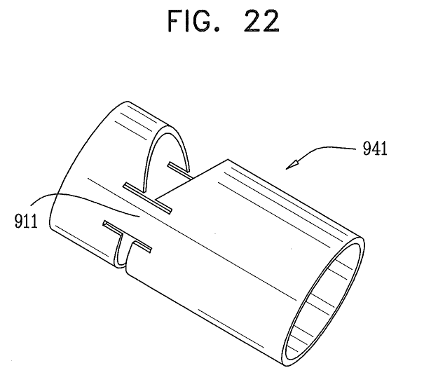

Reference is now made to FIG. 22, which is a schematic illustration of a plastic hinge 941, which is used for joint 911 of device 910, in accordance with some applications of the present invention. For some applications, using a plastic hinge for joint 911, enables aim 912 to be coupled to shaft 922 at a position that is closer to distal tip 916 of device 910, than if a pin is used for joint 911, as is shown in FIGS. 21A-B.

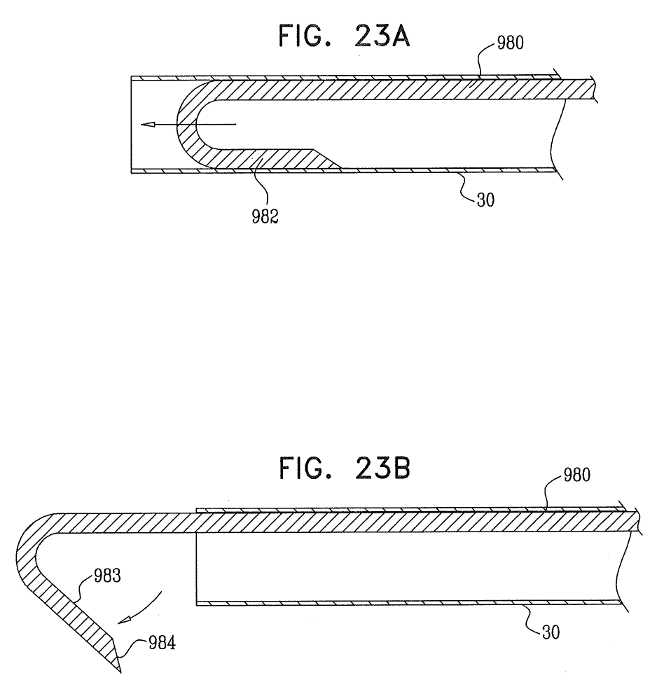

Reference is now made to FIG. 23A-B, which are schematic illustrations of a prostate capsule cutting device 980, in accordance with some applications of the present invention. For some applications, the cutting device is made of a shape-memory material, such as nitinol. The device is configured such that during insertion through introduction device 30, a proximal-facing distal portion 982 of the device is constrained by the introduction device. Upon passing out of the distal end of the introduction device, distal portion 982 opens automatically, based on material properties of the distal portion, as shown in FIG. 23B. A penetration element 984 of the distal portion, for example, a sharp tip of the proximal facing portion penetrates tissue, for example, tissue of the prostate capsule. For some applications, the penetration element penetrates the tissue automatically by the distal portion opening while the distal portion is adjacent to the tissue. Subsequent to the penetration of the tissue, a cutting element 983 (for example, a sharp edge of the proximal-facing portion, and/or a monopolar or bipolar electrode) is used to cut the prostate capsule or the prostate, typically, in accordance with the techniques described herein. In particular, cutting element 983 cuts tissue as cutting device 980 is pulled in a proximal direction. For some applications, the cutting element, or a different region of proximal-facing distal portion 982, is configured to coagulate the tissue. For example, the proximal-facing distal portion may comprise a single monopolar electrode, or bipolar electrodes for coagulating, and/or cutting the tissue.

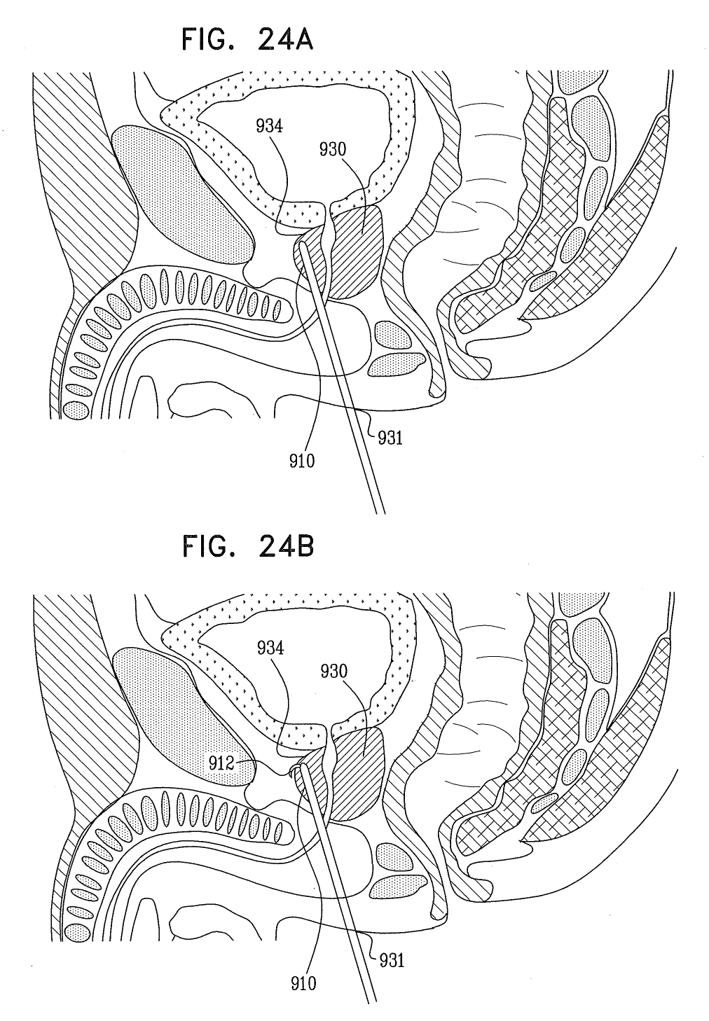

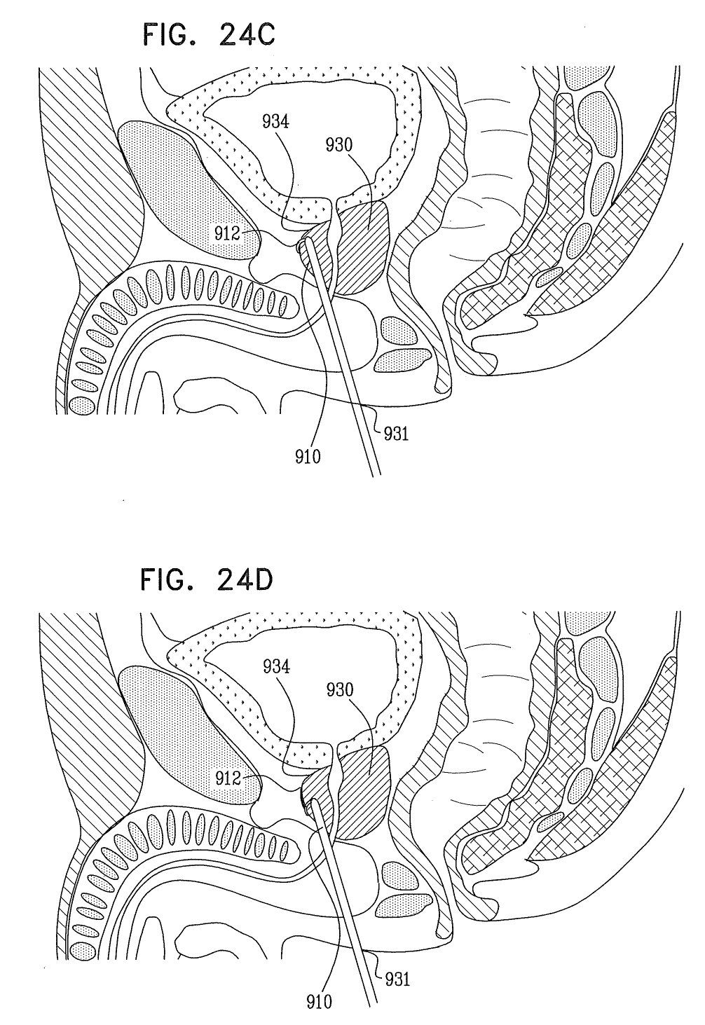

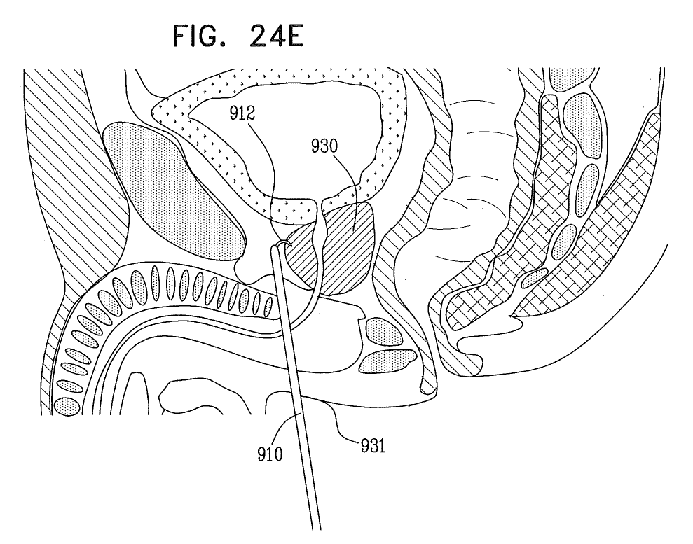

Reference is now made to FIGS. 24A-E, which are schematic illustrations of device 910 incising and/or excising the prostate capsule and/or prostate tissue, in accordance with some applications of the present invention. For some applications of the present invention, the procedure is performed while the patient is in the lithotomy position, under general/spinal/local anesthesia, and/or sedated. For some applications, the procedure is imaged, using, for example, transrectal ultrasound. Although applications are described with respect to FIGS. 24A-E according to which device 910 is used, the scope of the present invention includes using device 980, and/or other devices described herein, to practice the described techniques, mutatis mutandis. Although applications are described with respect to FIGS. 24A-E according to which a device is inserted transperineally, the scope of the present invention includes practicing the described techniques using a transabdominal, and/or a transurethral approach, mutatis mutandis.

In FIG. 24A, device 910 is shown being introduced into prostate 930 transperineally, while its arm 912 is closed. For some applications of the invention, two access ports 931 are formed (although only one port can be seen in FIG. 22A, since FIG. 22A shows a lateral view). One or more devices 910 are positioned in an upper, anterior portion of the prostate, close to capsule 934, for example, 1 mm to 10 mm, e.g., 1 mm to 4 mm, from the interior portion of capsule 934. For some applications, each access port 931 is of 2 mm to 6 mm diameter. Typically, in accordance with such applications, the outer diameter of the shaft of device 910 is between 1 mm and 5 mm. For some applications, two devices 910 are placed approximately parallel to each other. For some applications, two devices 910 are located at “10 o'clock” and at “2 o'clock” positions. Alternatively, a different number of access ports (and, accordingly, or otherwise, a different number of capsule incisions) are used. For some applications, one or more devices 910 are positioned differently with respect to the prostate.

Subsequent to positioning device 910 (as is verified by an imaging device, in accordance with some applications), button 920 (shown in FIG. 21A) of device 910 is pressed, and arm 912 protrudes from device shaft 922, into the prostate capsule 934 (i.e., from the prostate tissue into and through the capsule), as shown in FIG. 24B. At this stage, while prostate capsule tissue (and, optionally, a small amount of prostate tissue) is located in the space between the open arm 912 and device shaft 922, arm 912 is at least partially re-closed, to grasp the tissue, as shown in FIG. 22C.

Subsequently, the physician retracts device 910, thereby cutting the capsule tissue from within the prostate, as shown in FIG. 24D. For some applications, coagulation is provided to seal blood vessels, thus reducing bleeding, for example, by a physician pressing a button and/or foot-pedal, as described hereinabove. For some applications, cutting and coagulating are performed in steps—firstly, while penetrating/cutting element 912 is pressed against the tissue, the physician presses the coagulation button, and then slightly pulls back device 910 to cut the capsule. Alternatively, subsequent to coagulating the tissue, the capsule is cut using a blade, such as blade 921, described with reference to FIG. 21A. This operation is typically repeated a few times, until sufficient tissue has been incised. For some applications, the incisions are performed along the prostate capsule curvature, from the upper prostate part towards its apex. For some applications, incisions are made to a depth of 1 mm to 10 mm

Excision and/or incision of prostate capsule is achieved with or without a guiding stylet, in accordance with respective applications of the present invention. When a guiding stylet is used, it may be introduced, for example, perineally, up to the distal (upper) part of the anterior prostate, under image guidance. Following stylet positioning, device 910 is advanced over the guiding stylet. For some applications, a device is introduced into the lower part of the prostate, and the incision direction is from the prostate apex towards the upper part of the prostate, device 910 being modified appropriately to facilitate the forward motion during cutting.

For some applications, device 910 is inserted transperineally (or via a different approach), and the capsule is approached from the outside of the capsule, as shown in FIG. 24E. The cutting and coagulation device 910 is operated similarly to the procedure described with respect to FIGS. 24A-D.

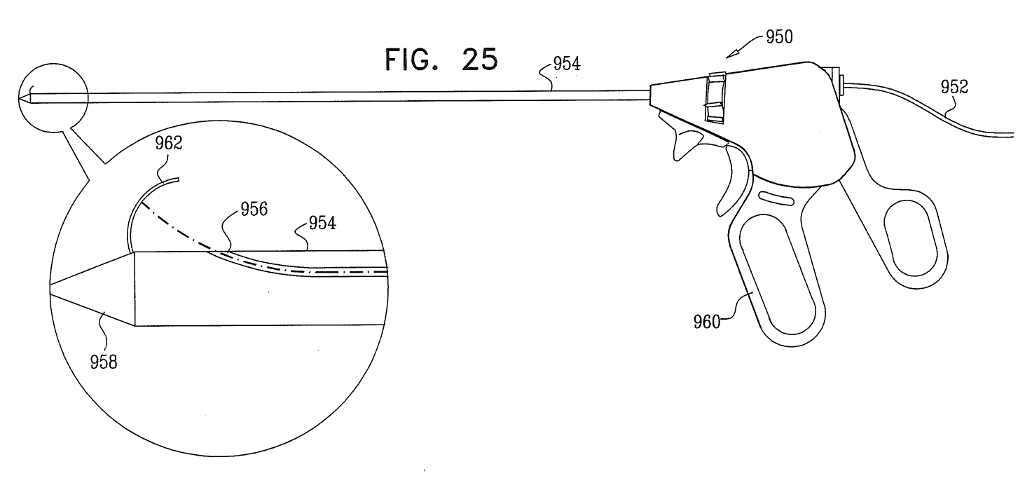

Reference is now made to FIG. 25, which is a schematic illustration of a laser device 950 for cutting and coagulating soft tissue, in accordance with some applications of the present invention. For example, device 950 may be based on green laser, red laser, or Nd:YAG technology. For some applications, the laser wavelength is in the range of 500 nm to 2,100 nm. A laser light guide 952 passes through the device shaft 954. For some applications, a distal end 956 of laser light guide 952 is angled, for example, upwards, thereby enabling incision of the prostate capsule from within the prostate. For some applications, device distal end 958 is pointed, to facilitate advancement of the device, and/or to prevent damage to tissue, while the device is introduced toward the prostate. For some applications, device 950 incorporates a handle 960 at its proximal part, and/or a button/foot pedal (not shown in FIG. 25) for device operation. For some applications, device 950 comprises a hook 962 at the distal end of the device. The hook is closed during device introduction toward the prostate. Following positioning of device 950, the hook is opened to penetrate the capsule tissue from within the prostate (or from outside of the prostate), in accordance with techniques described herein. For some applications, the hook is then at least partially re-closed, so that the tissue to be cut and/or ablated is gripped between hook 962 and a device shaft 954 (for example, using techniques described herein), in order to position the tissue in the path of the laser light guide distal end 956. For some applications, hook 962 shields surrounding tissue from the laser.

For some applications, device 950 comprises a drainage tube or tubing system, for draining out of the patient's body vapors generated during the operation of device 950. Alternatively or additionally, device 950 comprises a tube or tubing system, which circulates water in the surrounding tissue, thereby cooling the surrounding tissue (not shown in FIG. 25).

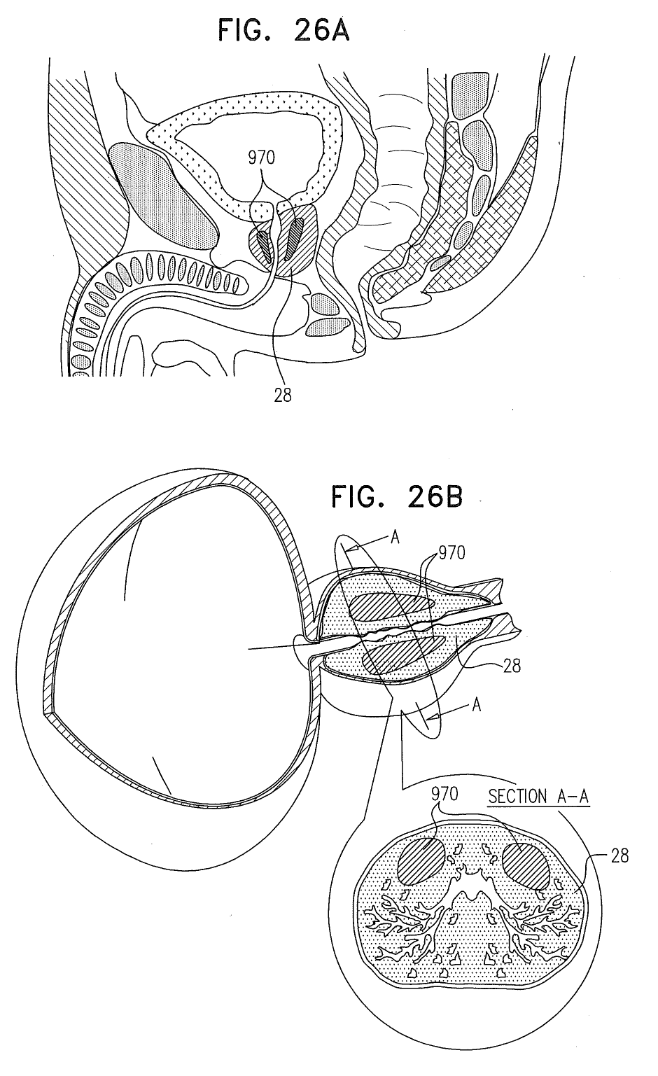

Reference is now made to FIGS. 26A-B, which are schematic illustrations of respective views of prostate 28, portions 970 of which have been resected percutaneously, transperineally, in accordance with some applications of the present invention. “Percutaneous,” as used in the specification and in the claims, refers to any medical procedure where access to inner organs or other tissue is done via a small puncture wound (e.g., using a needle and/or a trocar), as opposed to using an “open” approach where inner organs are exposed (e.g., as when using a scalpel). Typically, the resection is performed on a subject who is identified as suffering from benign prostatic hyperplasia. For some applications, the resection is performed on a subject identified as suffering from focal prostatic cancer.

Typically, in order to perform the transperineal resection, the subject is placed in the lithotomy position. A transrectal ultrasound probe is placed in the subject's rectum. The perineum is prepared for the procedure, for example, by cleaning the perineum. Under ultrasound guidance, a needle or other working sleeve (optionally, with a stylet that is later removed), is passed percutaneously, through the perineum, on either side of the midline, into the prostate. Typically, the needle is inserted while avoiding injury to the subject's urethra, bladder neck and prostate capsule (except for the access port of the prostatic capsule). Further typically, the needle is inserted without contacting the urethra. For some applications, the needle diameter is less than 6 mm, for example, less than 4 mm, in order to reduce damage to the tissue.

In accordance with respective applications, the needle is positioned without the use of needle-positioning equipment, or the needle is positioned using needle-positioning equipment, for example, a gridded plate attached to the table on which the patient lies. Typically, using various planes of view of the ultrasound, the needle is advanced to a sub-capsular position adjacent to the bladder neck. For some applications, a Foley catheter is placed in the subject's urethra and bladder, in order to facilitate visualization of the bladder neck and urethra.

Subsequent to the needle having been appropriately positioned, a resection tool is advanced into this position, optionally via the needle, and is used to resect (and, optionally, coagulate) and remove prostatic tissue. For some applications, a resection tool is inserted into the prostate directly, i.e., not via the needle. The resection is performed along a plane leading from the bladder neck toward the prostatic apex stopping at the level of the endopelvic fascia, as shown. Typically, the resection is performed while avoiding injury to the subject's urethra, bladder neck and prostate capsule (except for the access port of the prostatic capsule). Further typically, the resection is performed without the resection tool contacting the urethra. Typically, avoiding contacting the urethra reduces the risk of causing urethral trauma and associated urinary tract infections or urethritis, urethral strictures, and bladder neck stenosis. Once the procedure has been accomplished on one side of the midline, a similar procedure is typically performed on the contralateral side of the midline, at the equivalent contralateral location.

For some applications, subsequent to the prostate having been resected, a tissue-collecting tool is inserted into the prostate (e.g., via the needle, or directly), and collects the resected tissue and removes the resected tissue from the subject's body. For some applications, a single tool is used to resect (and, optionally, coagulate) the prostate and to remove the resected tissue. Typically, the tissue collection is performed while avoiding injury to the subject's urethra, bladder neck and prostate capsule (except for the access port of the prostatic capsule). Further typically, the tissue collection is performed without the tissue-collecting tool contacting the urethra.

In accordance with respective applications, the portion of the prostatic capsule that overlies the resected tissue is, or is not incised, resected, and/or coagulated. For some applications, the capsule is resected and/or incised from within the prostate, for example, through the aforementioned needle. For some applications, the same resection tool is used for prostatic capsule resection and/or incision that is used for prostatic tissue resection. For some applications, a different instrument is used for resecting and/or incising the prostatic capsule, for example, an instrument that uses mechanical cutting, RF energy, laser and/or other technology. For some applications, for example, under transrectal ultrasound guidance, the prostatic capsule is incised and/or resected (and coagulated) at its anterior part, in order to avoid damage to the neurovascular bundles which are located more dorsolaterally. For example, two relatively parallel, longitudinal incisions may be performed at the 2 o'clock and o'clock positions along the anterior capsule tissue, from the upper part of the capsule toward the prostate apex.

For some applications, the prostate capsule is approached from outside the prostate, in order to incise and/or resect the prostatic capsule. For some applications, for example, under transrectal ultrasound guidance, a needle and/or a resection tool are introduced percutaneously, transperineally into the Retzius space, i.e., the retropubic space above the prostate. For some applications, prostate capsule incision and/or resection is performed with or without resection and removal of prostatic tissue (for example, a prostatic adenoma).

Typically, portions 970 include anterior portions of the prostate. For some applications, portions 970 include one or more posterior portions of the prostate that are resected transperineally and/or transrectally.

For some applications, blood vessels are identified and avoided, for example, using transrectal ultrasound in an echo-Doppler mode.

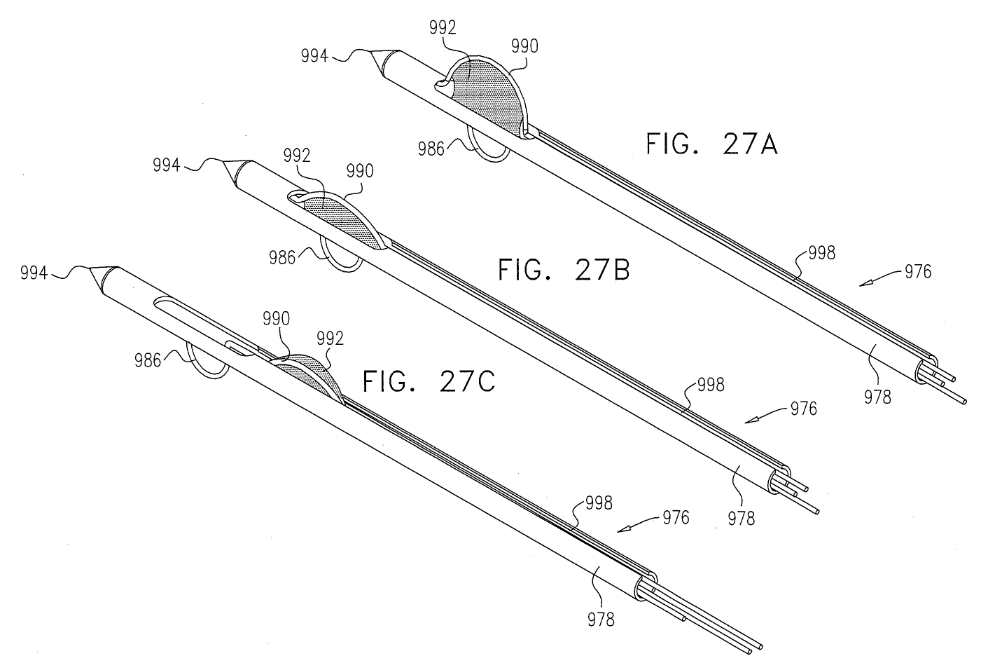

Reference is now made to FIGS. 27A-C, which are schematic illustrations of a tissue resection device 976 in respective configurations, in accordance with some applications of the present invention. Device 976 includes a longitudinal shaft 978, having a rotating cutting-loop 986 at its distal end and a handle at its proximal end. For some applications, device 976 includes a tissue-collecting loop 990 filled with a net 992 (e.g., braided material) at the distal end of device 976, as described hereinbelow. For some applications, cutting loop and tissue-collecting loop are disposed on separate shafts from one another, and are used simultaneously with one another, or at different times from one another. Cutting (and, optionally, coagulation) of the prostate is achieved using, for example, RF energy, mono-polar and/or bi-polar techniques, laser, cold knife, or other technologies (e.g., the Harmonic Scalpel®). Device 976 has a pointed tip 994 to facilitate insertion of the device into the subject's prostate.

For some applications, during insertion of device 976 into the subject's prostate, cutting-loop 986 is retracted into the shaft. Subsequent to the distal end of the shaft 978 having been inserted into and properly positioned within the prostate, a user releases cutting-loop 986 from a slot (not shown) in shaft 978, for example, by pressing a button (not shown) located on the device handle.

For some applications, the entire shaft 978, including cutting-loop 986, is rotated during operation of the loop, i.e., during resection of the prostate by the cutting-loop. For some applications, the shaft is rotated, while being moved proximally, such that the loop makes a helical incision in the prostate. Alternatively, during operation of the cutting-loop, the shaft remains stationary and the cutting-loop rotates, moves proximally, or rotates while moving proximally. For some applications, the slot from which the cutting loop is released spirals along the shaft, and, during operation of the cutting-loop, the cutting-loop moves through the slot, helically, without rotation of shaft 978 and/or without rotation of the device handle. Alternatively, shaft 978 is driven to rotate, while the device handle does not rotate. For some applications, rotational motion of the cutting-loop is actuated by means of linear movement of a trigger-like element on the device handle. For some applications, device 976 (e.g., loop 986 of device 976) coagulates tissue before, simultaneously with, or subsequent to cutting of the tissue. For example, a foot pedal (not shown) that is coupled to device 976 may actuate the device to coagulate the tissue.

For some applications, shaft 978 has a diameter of less than 6 mm, or less than 4 mm (e.g., 2 mm-3 mm), the diameter being sized so as to reduce damage to tissue. Typically, the volume of tissue that is resected from the prostate comprises a substantial portion of the total prostate volume, e.g., more than 5%, or more than 10% (for example, about 20%) of the prostate volume is resected, in order to create a cavity in the prostate and relieve pressure on the urethra.

For some applications, a height of cutting loop 986 from shaft 978 is 2 mm to 10 mm, and the width of the cutting loop is similar in size, or greater. In accordance with respective applications, the cutting loop is round, semicircular and/or oval.

For some applications, device 976 is introduced into the prostate, and then operated and controlled, via a small diameter cannula. For some applications, the device is removed via the cannula.

For some applications, during operation of cutting-loop 986, the cutting-loop is repeatedly retracted into and released from the shaft. Typically, this action results in several pieces of tissue being cut from the prostate instead of a single, continuous piece of tissue. For some applications, the pieces of tissue are removed by flushing the prostate with a flushing liquid, such as sterile saline, or glycine.

For some applications, a rod is coupled to shaft 978, as described in U.S. Provisional Patent Application 61/185,660 to Goldwasser, filed Jun. 10, 2009, which is incorporated herein by reference. For example, the rod may be parallel to the device shaft and connected to the device handle. For some applications, the distal end of the rod is curved toward cutting-loop 986. While the prostate tissue is resected by cutting-loop 986, the resected tissue is threaded onto the rod. Alternatively or additionally, a vacuum is used to suck the resected tissue out of the subject's body.

As described hereinabove, for some applications, device 976 includes a tissue-collecting loop 990 filled with a net 992 (e.g., braided material) at the distal end of device 976. The net may be made of, for example, nylon, and/or polyethylene, such as high-molecular weight polyethylene. For some applications, net 992 is made from Dyneema® fibers. Typically, the material out of which net 992 is made is chosen so as to enable net 992 to be very thin, such that device 976 can be introduced into the subject's body in a small diameter configuration. Further typically, the mesh density of the net is such that it is able to collect even small pieces of tissue, but it allows the flow of liquids, e.g., blood, therethrough.

For some applications, tissue-collecting loop 990 and net 992 are retracted into a slot 998 in shaft 978 during insertion of device 976 into the subject's prostate (and, optionally, during removal of device 976 from the prostate). A user actuates the tissue-collecting loop and the net to protrude from the shaft, for example, by operating a button on the device handle. Net 992 is used to collect prostate tissue that has been resected.

FIGS. 27A-C show three configurations of the tissue-collecting loop and the net with respect to the shaft. The three configurations that are shown are respective stages of the return of the tissue-collecting loop and the net into the shaft, starting with the top configuration in which the tissue-collecting loop and the net have not yet been withdrawn into the shaft at all. It is noted that the tissue-collecting loop is typically returned into the shaft when there is resected tissue inside the net, although the resected tissue is not shown in FIGS. 27A-C.

For some applications, following tissue resection, and during a tissue collecting phase of the operation of device 976, the entire shaft of the device, including loop 990, is rotated while being moved proximally, and the tissue previously cut is collected in the net and removed. Alternatively, the shaft remains stationary, and the tissue-collecting loop and the net are rotated, are moved proximally, and/or are rotated while being moved proximally. For some applications slot 998 in shaft 978 of device 976 is a slot that spirals along the shaft, and, during operation of the tissue-collecting loop, the tissue-collecting loop moves through the slot, helically, without rotation of shaft 978 and/or without rotation of the device handle. Alternatively, shaft 978 is driven to rotate, while the device handle does not rotate. For some applications, rotational motion of the tissue-collecting loop is actuated by means of linear movement of a trigger-like element on the device handle.

For some applications, cutting loop 986 and tissue-collecting loop 990, which is coupled to net 992, are integrated into a single loop. In such applications, net 992 is made from appropriate material, for example, a material that can withstand heat (such as, carbon) and/or that does not conduct electrical current.

For some applications, device 976 comprises both cutting loop 986 and tissue-collecting loop 990, as shown in FIGS. 27A-C. Thus, during device rotation, cutting loop 986 cuts (and coagulates) the tissue, and tissue-collecting loop 990 collects the resected tissue. For some applications, the two loops are adjacent to each other (configuration not shown). Alternatively, as shown in FIGS. 27A-C, the two loops are disposed with a rotational offset around shaft 978, e.g., at an angle of 180 degrees from each other as shown in FIGS. 27A-C. For some applications, the distance between the two loops is chosen to ensure that the net will not be excessively heated during operation of the device, for example, if the net is made from a material that cannot withstand the high temperature.

For some applications, tissue-collecting loop 990 and/or net 992 are exchanged during a procedure as described in U.S. Provisional Patent Application 61/185,660 to Goldwasser, filed Jun. 10, 2009, which is incorporated herein by reference. For example, following resection of tissue using cutting loop 986, collection of the resected tissue is performed using tissue-collecting loop 990 and net 992. Subsequently, if additional tissue cutting is desired, the user removes from shaft 978 the tissue-collecting loop 990 and net 992 with resected tissue inside the net (for example, via slot 998), and performs addition tissue resection. Then, another tissue-collecting loop and net are loaded onto the device and used for removal of the cut tissue.

For some applications, as shown, tissue-collecting loop 990 is substantially bigger than cutting loop 986. Typically, this ensures that all of the resected tissue is collected and removed from the patient body. Alternatively, the cutting loop and the tissue-collecting loop are of relatively similar sizes. For some applications, a height of tissue-collecting loop 990 from shaft 978 is 10 mm to 20 mm, and the width of the tissue-collecting loop is similar in size, or greater. In accordance with respective applications, the tissue-collecting loop is round, semi-circular, and/or oval.

For some applications, cutting loop 986 and tissue-collecting loop 990 are disposed on the distal ends of two different devices. Following resection of the tissue, using, for example, a device that includes a cutting loop, a tissue-collecting device (including tissue-collecting loop 990 and net 992) is introduced into the prostate (typically, via the same port and, optionally, via the same needle). When the distal end of the tissue-collecting device is suitably positioned inside the subject's prostate, the user presses a button of the tissue collecting device and the tissue-collecting loop and the net emerge from a slot 998 of the shaft of the tissue-collecting device, as described hereinabove.

Subsequently, for some applications, the entire shaft of tissue-collecting device, including loop 990, is rotated while being moved proximally, and the tissue previously cut is collected in the net and removed. Alternatively, the shaft remains stationary and the tissue-collecting loop and the net are rotated, are moved proximally, and/or are rotated while being moved proximally. For some applications, a slot in the shaft of the tissue-collecting device is a slot that spirals along the shaft, and, during operation of the tissue-collecting loop, the tissue-collecting loop moves through the slot, helically, without rotation of the shaft and/or without rotation of the handle of the tissue-collecting device. Alternatively, the shaft is driven to rotate, while the handle of the tissue-collecting device does not rotate. For some applications, rotational motion of the tissue-collecting loop is actuated by means of linear movement of a trigger-like element on the handle of the tissue-collecting device.

For some applications, the tissue-collecting device is introduced into the prostate and/or is operated and controlled via a small diameter cannula. For some applications, the tissue-collecting device is removed from the prostate via the cannula.

For some applications, cutting loop 986 and tissue-collecting loop 990 operate simultaneously. Alternatively, cutting (and, optionally, coagulation) is performed by cutting loop 986, and, subsequently, the tissue is collected using tissue-collecting loop 990.

Experiments were performed by the inventors, in which canine prostatic tissue and beef were resected using a rotating loop, as described hereinabove. Subsequently, a tissue-collecting device, as described hereinabove was used to collect the resected tissue. Subsequent to the tissue having been resected, the tissue was dissected, in order to determine the efficacy of the tools in resecting tissue and in collecting the resected tissue. The dissected tissue indicated that a cavity having the desired dimension (relative to the cutting loop size) was formed. Furthermore, the tissue-collecting device effectively removed the resected tissue. Thus, the devices described herein may be used for tissue (e.g., prostate tissue) mass reduction, leading, in the case of prostate tissue mass reduction, to pressure relief on the urethra.

It is noted that some of the devices described herein generate heat while operating. It is hypothesized by the inventors that tissue removal and controlled heat generation may neutralize and/or damage the intrinsic prostate nerves and smooth muscle cells of the prostate gland, resulting in reduction in tone of these cells, which will lead to relief of pressure on the urethra. Typically, the devices described herein operate so as not to cause excessive thermal damage, and so as to remove resected tissue from the subject's body. The inventors hypothesize that the tissue healing process resulting from using the techniques described herein is shorter (for example, with less edema and inflammation) than with other techniques, such as RF ablation technique (e.g., trans-urethral needle ablation), in which (a) the thermal damage caused to the tissue is greater (b) the treated tissue is not removed, and (c) damage to the urethra may occur.

For some applications, a device that is generally similar to device 976 described with reference to FIGS. 27A-C is used. However, instead of or in addition to cutting loop 986 and/or tissue collection loop 990, the device has an expandable structure capable of cutting (and optionally coagulating) tissue, at its distal end, as described in U.S. Provisional Patent Application 61/185,660 to Goldwasser, filed Jun. 10, 2009, which is incorporated herein by reference.

It is noted with respect to the applications described herein, that following tissue resection, the distal portion of device 976 and/or another device described herein may have a relatively large diameter (for example, compared to the device shaft) due to the resected tissue entrapped within the distal portion of the device. For some applications, device removal can be performed such that the distal portion of the device does not pass through a cannula (working sleeve) through which the device was inserted into the prostate. Rather, the distal portion may be removed by passing directly through the lumen created in the tissue, during insertion of the device. As the tissue is elastic, it is compliant with the larger diameter distal portion, and facilitates its passing through the tissue.

For some applications, device 976 and/or another device described herein is not introduced via a cannula. Alternatively, the device is introduced through a cannula; however, during removal of the device, the distal portion of the device is not retrieved into the cannula. Rather, the cannula with the device are pulled back and removed from the subject's body at the same time.

For some applications, the subject's prostate is flushed with liquid (e.g., with sterile saline, or glycine) and the liquid is aspirated, during the procedure, for example, via a cannula through which resection device 976 and/or another device described herein is inserted. For some applications, the cannula includes two lumens to provide for circulation (i.e., introduction and aspiration) of liquid. Alternatively or additionally, an additional needle (optionally, defining two lumens) is introduced, optionally, via the cannula, for liquid introduction and aspiration. For some applications, the prostate tissue resection procedure is performed via two cannulae, and one or both of these cannulae is also used for introduction and/or aspiration of a flushing liquid.

For some applications, a cooling liquid is introduced into the prostate. For some applications, a thermocouple is located on the distal portion of resection device 976 and/or another device described herein. Based on the temperature measured by the thermocouple, the flow rate of the cooling liquid and/or the liquid temperature is changed, in order to maintain the temperature of the region of prostate that is being treated within a predetermined range.

For some applications, cutting loop 986 and/or a wire of device 976 conducts energy, for example, in order to cut and/or coagulate prostatic tissue. For some applications, the device components that do not conduct energy are electrically insulated and/or are not connected to an electrical source.

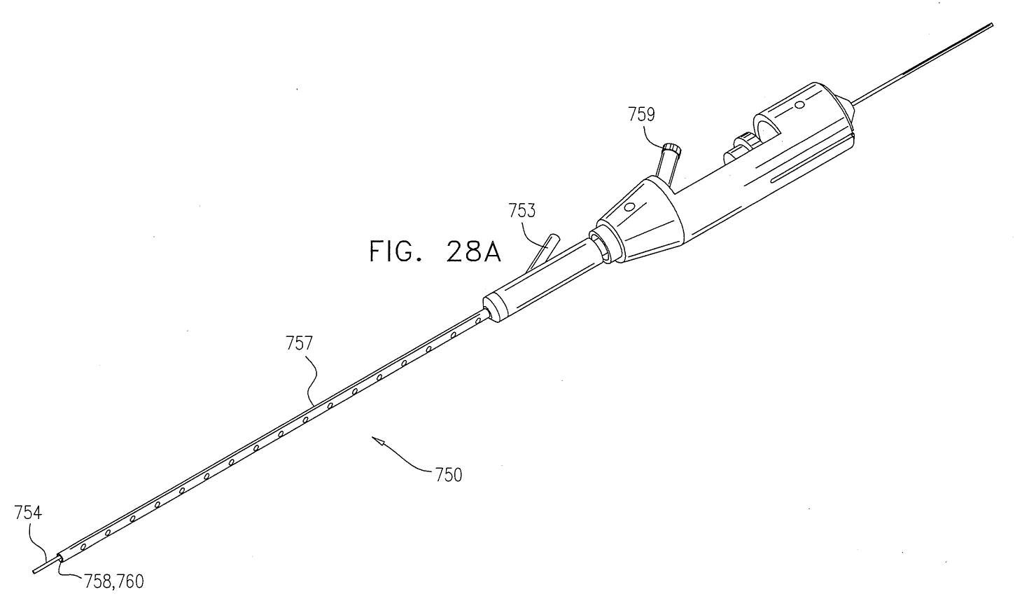

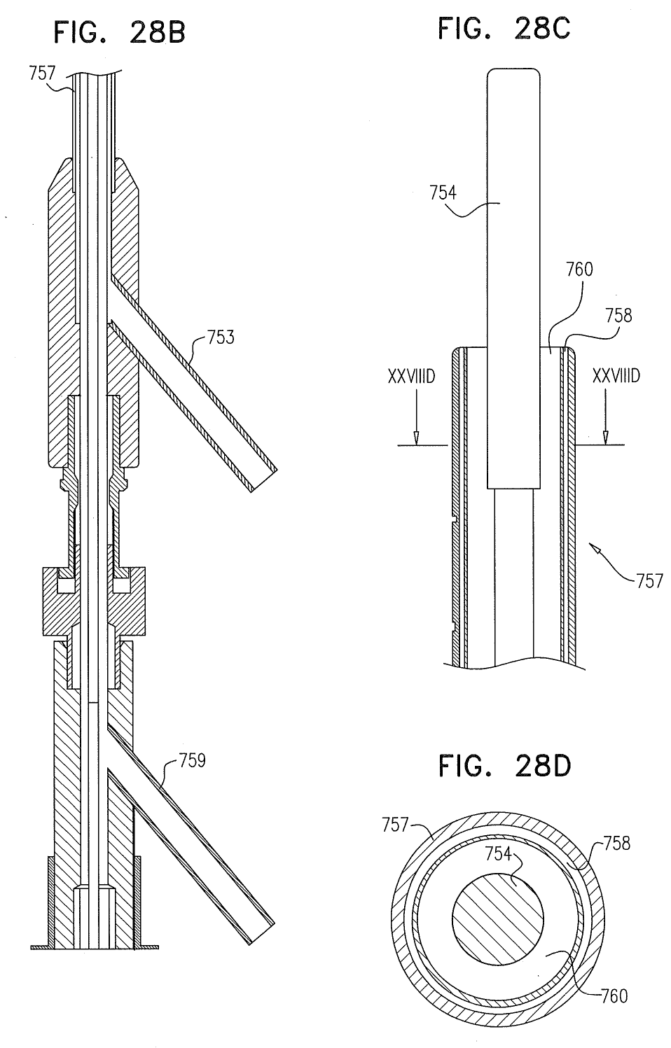

Reference is now made to FIGS. 28A-D, which are schematic illustrations of a laser device 750 (e.g., green laser, or a different frequency laser), which thermally ablates tissue of the prostate, in accordance with some applications of the present invention. The figures show respectively a three-dimensional schematic illustration of the device (FIG. 28A), a longitudinal cross-section of a proximal portion of the device (FIG. 28B), a longitudinal cross-section of a distal portion of the device (FIG. 28C), and a cross section of the distal portion of the device the cross-section being perpendicular to the longitudinal axis of the device (FIG. 28D). For some applications, a shaft 757 of device 750 defines longitudinal lumens 758 and 760, through which liquid (e.g., sterile saline or glycine) is, respectively, introduced and aspirated. The liquid is introduced via a port 753 at the proximal end of the device. The liquid that is aspirated is typically suctioned out from the proximal end of the device via an aspiration port 759.

For some applications, the liquid is a cooling liquid that decreases the temperature in the vicinity of the distal end of laser fiber 754 and/or in the surrounding tissue, thereby minimizing damage to the fiber and/or the tissue. For some applications, the liquid is circulated around the laser fiber. For some applications, in addition to the liquid being aspirated from the prostate, the products of the ablation of the prostatic tissue by the laser are aspirated. Thus, typically (as shown, for example, in FIG. 28C), aspiration lumen 760 has a larger diameter than that of the liquid inlet lumen 758, in order to facilitate the passage of the ablation products through lumen 760, out of device 750 and the subject's body. For some applications, in addition to acting as a cooling fluid, the liquid acts as a flushing liquid for flushing the ablation products from the prostate.

For some applications, device 750 provides a constant distance between laser fiber 754 (via which the laser light is transmitted) and the treated tissue. For example, the device may include a recess between a portion of the laser fiber (e.g., the tip of the optic fiber) adjacent to the recess and the tissue-contacting surface of shaft 757. For example, a laser device that defines a recess may be used as described in U.S. Provisional Patent Application 61/185,660 to Goldwasser, filed Jun. 10, 2009, which is incorporated herein by reference. In accordance with respective applications, the laser fiber emits a laser beam generally in the direction of the longitudinal axis of shaft 757, or at an angle thereto.

For some applications, a thermocouple is located on the distal portion of laser device 750. Based on the temperature measured by the thermocouple, the flow rate of the cooling liquid and/or the liquid temperature is changed, in order to maintain the temperature of the region of the prostate that is being treated within a predetermined range.

For some applications, device 750 comprises a handle at its proximal end. For some applications, device 750 is introduced into the prostate, and operated and controlled (and, optionally, removed) via a small diameter cannula.

It is noted that in contrast to prior art laser devices, which are introduced into a natural orifice, and advanced through a natural lumen of the body (e.g., the urethra), device 750 is typically introduced via a non-natural orifice and is advanced through a non-natural lumen. It is noted that the scope of the present invention includes introducing device 750 via a non-natural orifice, and advancing device 750 through a non-natural lumen in order to access an organ of the subject other than the prostate, e.g., the subject's liver or kidney. Device 750 is used to ablate the subject's kidney or liver in a generally similar manner to that described herein with respect to the ablation of the subject's prostate by the device, mutatis mutandis.

Typically, device 750 is transperineally inserted into the subject's retropubic space (i.e., the space between the posterior surface of the pubic bone and the anterior surface of the prostate capsule). For some applications, insertion of the device 750 into the retropubic space is guided by transrectal ultrasound. The device thermally ablates prostatic tissue. Typically, due to the ablation, heat and gases are generated. Device 750 generates fluid flow at the region of the ablation, thereby cooling the region, and/or removing the ablation products. Typically, shaft 757 of the device includes laser fiber 754 via which the laser is transmitted, inlet lumen 758, and aspiration lumen 760, and the diameter of the shaft is less than 8 mm. For example, the diameter of the shaft is less than 5 mm, and/or more than 3 mm, e.g., 3 mm to 5 mm.

For some applications, one or more of the devices described herein for incising or resecting the prostate, and/or for incising and/or resecting the prostatic capsule is transperineally inserted into the subject's retropubic space. For some applications, insertion of the device into the retropubic space is guided by transrectal ultrasound.

It is noted with respect to all of the devices described herein that, for some applications, the device includes markings that enhance its visualization under ultrasound guidance. For example, the device may have a rough surface and/or a porous coating, which improves the reflection of the ultrasound waves from the device and thus enhances device recognition relative to surrounding tissue or fluids under ultrasound imaging. For some applications, the described effect is in accordance with the effects described in the following article and patent, which are incorporated herein by reference:

“In-Vitro Visualization of Biopsy Needles with Ultrasound: A Comparative Study of Standard and Echogenic Needles Using an Ultrasound Phantom,” by Hopkins R. E. et al., Clin Radiol 2001; 56: 499-502; and

U.S. Pat. No. 7,229,413 to Violante et al.

For some applications, the procedures described herein are performed under ultrasound guidance or under the guidance of other imaging devices, such as MRI, CT, and/or an optical fiber inserted into the space created within the prostate by tissue being resected.

For some applications, the procedures described herein are aided by navigation systems and/or are computer-aided and/or robotic-aided. For example, such systems may be used to guide introduction and/or operation of the devices described herein

Although applications are described according to which the prostate is resected via a percutaneous transperineal approach, the scope of the present invention includes resecting the prostate via any one of the following other percutaneous approaches:

suprapubic approach;

retropubic approach;

transrectal approach;

laparoscopic approach; and

transurethral approach.

For some applications, device 750, device 976, and/or a different device described herein is inserted through the urethra, and the urethra is only pierced at the area of the prostatic urethra, but not elsewhere along the urethra, to enable introduction of the device into the prostatic lobe. Typically, in prior art transurethral resection procedures, the prostatic urethra is resected, and therefore, relatively substantial damage is caused to the urethra. Typically, in prior art transurethral needle ablation procedures, there is less damage to the urethra than in transurethral resection procedures, but tissue is not removed. Furthermore, transurethral needle ablation procedures may result in urethral tissue becoming damaged by being heated.

For some applications, the prostate is resected without the prostate capsule being incised, apart from a small access port through which the resection device is inserted. The inventors hypothesize that in such applications, if bleeding occurs during the resection of the prostate, it will be contained within the prostate. (This is a similar effect to the effect of bleeding that is generated during saturated transrectal prostate biopsies being contained within the prostate, as is known in the prior art). By contrast, in some prior art transurethral resection procedures, the urethra may become damaged and bleeding may proceed into and through the urethra.

Typically, the techniques and devices described herein are used for the treatment of BPH. However, they are not limited to this indication. For example, the techniques and devices described herein may be used for prostate cancer treatment. Typically, if cancerous tissue is located in only one of the prostate lobes, such as in focal prostatic cancer, it is possible to resect only this tissue, with or without the prostate capsule, using the techniques and devices described herein.

For some applications, following prostate tissue resection (and, optionally, coagulation), for example, using device 976 having cutting loop 986, described with reference to FIGS. 27A-C, the device is retrieved from the subject's body. Subsequently, a laser device (e.g., laser device 750) is introduced through the same port (optionally, via a cannula) into the prostate, to perform coagulation and/or additional coagulation.

In an alternative application of the invention, a laser device (e.g., laser device 750) is first introduced into the prostate tissue for tissue ablation and coagulation. The device is removed and then a resection device (e.g. device 976) is inserted via the same access port in the capsule (and, optionally, via a cannula), to resect (and optionally remove) non-viable and/or necrotic tissue generated by the laser procedure. For some applications, performing the procedures in this order (i.e., ablation and coagulation by laser, followed by resection) reduces bleeding, as tissue resection is conducted following tissue coagulation.

For some applications, heating and/or ablation of the prostate tissue is performed remotely (e.g., from outside the subject's body), for example, using high-intensity focused ultrasound, in combination with localization tools, such as MRI or other guidance technologies (e.g., the technology used in the Exablate® device). Subsequently, a resection tool (e.g., device 976) is inserted into the prostate tissue, to resect (and remove) non-viable and/or necrotic tissue generated following remote-heating (e.g., the high-intensity focused ultrasound) operation. Thus, this procedure also provides for resection following coagulation of the tissue. For some applications, the procedure described in this paragraph is not limited to treating the prostate for benign prostatic hyperplasia, but may also be performed for treating other conditions and tissue, including cancerous and metastatic tissue, in the liver, kidney, uterus, etc.

For some applications, prior to prostate tissue resection, ablation, and/or removal, a vasoconstrictor substance (e.g., adrenaline or lidocaine) or an anesthetic (e.g., lidocaine) is locally administered to the prostate tissue (e.g., by injection). For some applications, the substance serves as a local anesthetic agent and, thus, relieves pain following the procedure.

For some applications, prior to prostate tissue resection, ablation, and/or removal, a blood vessel occluding agent is injected locally into the tissue of the prostate, to prevent bleeding following the procedure.

For some applications, one or more of the procedures described herein are performed percutaneously, transperineally, for example while the patient is in the lithotomy position. For some applications, transperineal access is performed via at least at one lateral portion of the urogenital triangle, relatively close to the triangle base. Optionally, transperineal access into the retropubic space is gained from a more posterior point, with a more acute access angle.

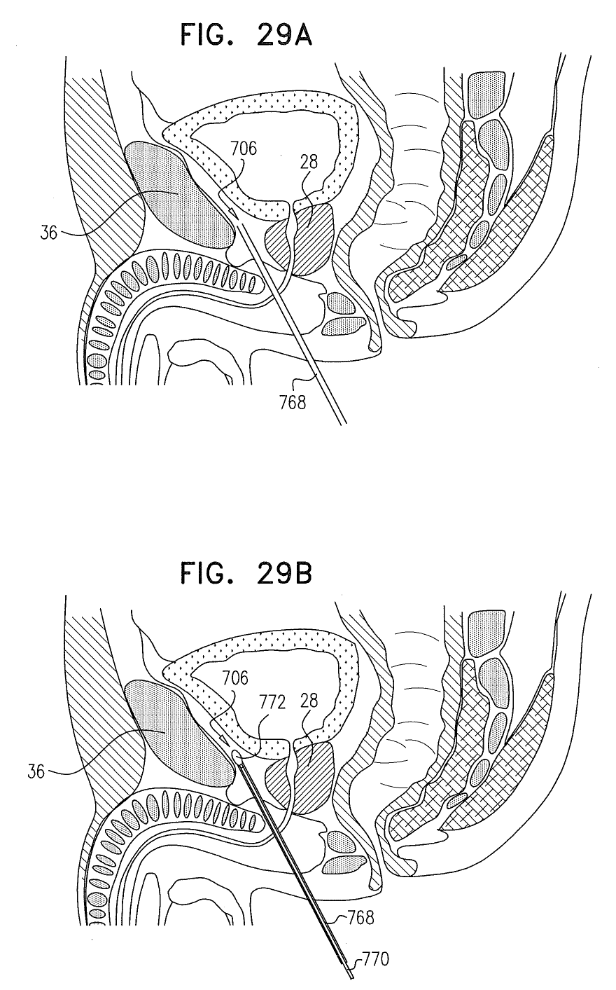

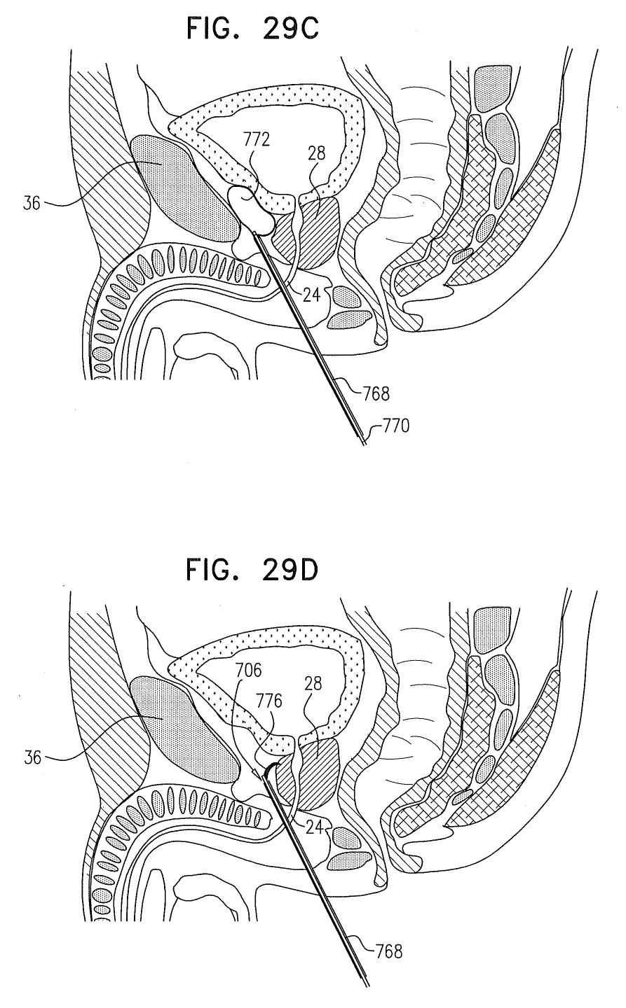

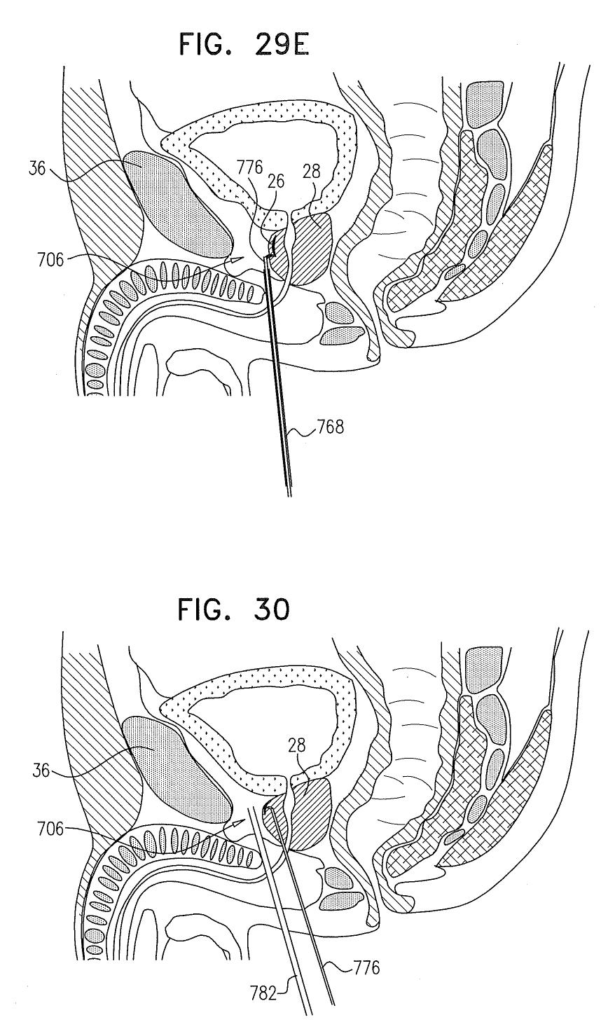

Reference is now made to FIGS. 29A-E, which are schematic illustrations of respective steps of a procedure for treating the prostate 28, in accordance with some applications of the invention.

As shown in FIG. 29A, at least one cannula 768 is introduced percutaneously, transperineally (e.g., through the perineal urogenital triangle), into retropubic space 706 (i.e., anteriorly to the prostate, between the prostate 28 and the pubic bone 36). Note that access is performed laterally to the midline (not shown in FIGS. 29A-E, which are lateral views). Optionally, insertion of the cannula is performed under ultrasound imaging (e.g., transrectal or transabdominal ultrasound imaging). For some applications, cannula 768 is part of an endoscopic device.

Following insertion of cannula 768 via an access port in the urogenital triangle (as described hereinabove), a balloon device 770 is inserted into the prostate via the cannula, as shown in FIG. 29B. (Alternatively, the balloon device is not separate from the cannula, but rather the cannula comprises an inflation tube of the balloon device.) As shown in FIG. 29C, retropubic space 706 between the pubic bone 36 and prostate 28 (i.e., adjacent to the anterior portion of the prostate) is enlarged, by inflating a balloon 772 of balloon device 770. Balloon 772 is inflated, by filling the balloon with a fluid such as water, saline, air, and/or carbon dioxide, as is known in the art. Typically, the enlargement of the retropubic space facilitates a procedure adjacent to the anterior portion of the prostate, for example, as described hereinbelow. For some applications, air and/or another fluid is directly introduced into the retropubic space, without the use of a balloon. For some applications, the inflation is performed under image guidance, for example using transrectal ultrasound.

For some applications, balloon 772 is designed to have a configuration and dimensions that comply with the anatomy of its designated location. For example, the balloon may have a round shape, elliptic shape, a curved (e.g., crescent) shape, rectangular shape, etc. Optionally, two balloons are introduced and inflated, one in the right portion of the retropubic space, and the other in the left portion. For some applications of the invention, balloon dimensions may be in the size of the retropubic area, or approximately half of the size of the retropubic area. Once the balloon is inflated, the retropubic space, between the pubic bone and the anterior prostate surface, is enlarged such that the depth of the space is more than 1 mm, and/or less than 5 mm, e.g., 1-5 mm, according to balloon inflation depth. For some applications, the depth of the retropubic space is enlarged to more than 5 mm, or less than 1 mm. For some applications, one or more of the balloons described hereinabove with reference to FIGS. 7A-C, 8A-F, and/or 10A-C is used.

For some applications, subsequent to its inflation, balloon 772 is deflated and removed. For some applications, the space created by the balloon inflation is filled, and optionally further inflated with a fluid, for example, carbon dioxide. For some applications, subsequently, a direct vision device, such as an endoscope, is introduced transperineally into retropubic space 706 through cannula 768. Subsequently, as shown in FIG. 29D (typically, under direct vision), a device 776 for incision, excision, and/or ablation of prostatic tissue is introduced. For some applications, cannula 768 is part of an endoscopic device, and device 776 is inserted via the cannula of the endoscopic device under endoscopic vision. Device 776 is used to cut the anterior portion of the prostate capsule, and optionally, to cut, resect, and/or ablate prostatic tissue, to relieve pressure in prostate 28, without damaging urethra 24, as shown in FIGS. 29D and 29E.

For some applications, the procedure is performed via a plurality of cannulae. For example, first and second cannulae may be inserted transperineally and contra-laterally from one another. For some applications, one or more of the cannulae is a cannula of an endoscopic device that provides endoscopic vision of the procedure.

For some applications, two longitudinal incisions are made in the anterior portion of the prostatic capsule 26, at the 10 o'clock and 2 o'clock positions, from the upper part of the prostate towards the prostate apex. Alternatively or additionally, prostate tissue is incised and/or resected. For some applications, a different number of incisions are made, and/or incisions are made at different locations of the prostatic capsule. For some applications, the incisions are made to be sufficiently wide as to reduce the possibility of adhesion (i.e., reclosure) of the incisions.

In accordance with respective applications, device 776 includes, for example, a diathermia device, a cold knife, a cauterizing tool, monopolar and/or bipolar RF devices, a laser device, a morcellation device, a microdebrider, a cryotherapy device, and/or an ablation device. For some applications, a plurality of the aforementioned devices are used for the procedure. For some applications, a device that includes a tissue collection member may additionally be used, in accordance with the techniques described hereinabove. For some applications, the procedure includes coagulation of blood vessels, as described hereinabove.

For some applications, a microdebrider is used that includes a tube with a blade at its distal end. Typically, the tube is coupled to a suction source, and the blade is operated via electrically powered motor. The microdebrider typically includes a control unit, which allows the operator to select the speed and the direction of blade rotation. For some applications a microdebrider provides relatively fast tissue resection and removal compared to other devices for tissue resection and removal. Typically, resected tissue becomes attached to the device due to the suction source. Therefore, the resected tissue is typically removed from the subject's body even when the procedure is performed under indirect vision (e.g., via ultrasound). For some applications, the microdebrider forms incisions that are sufficiently wide as to reduce the possibility of adhesion (i.e., reclosure) of the incisions.

For some applications, the microdebrider includes a diathermy portion, for coagulating tissue. Typically, for such applications, portions of the device are insulated. Alternatively, following mechanical resection of the tissue using the microdibrider, the microdebrider is withdrawn and a device capable of tissue coagulation (e.g., diathermia device) is introduced to seal the blood vessels.

Reference is now made to FIG. 30, which is a schematic illustration of device 776 being used to incise and/or resect prostate capsule tissue and/or prostatic tissue. The device is introduced into prostate 28, for example, into the anterior part of the prostate and/or into the right lobe and/or the left lobe of the prostate. The device is used to resect prostate tissue and/or to cut the prostate capsule from within the prostate. For some applications, an endoscope 782 is introduced transperineally into the retropubic space following inflation of a balloon as described above. Subsequently, device 776 for cutting, resection, and/or ablation is introduced percutaneously, transperineally into the prostate, directly, or via a cannula which was previously introduced into the prostate. The use of the endoscope that provides direct vision may be beneficial, for example, if it is necessary or desirable to apply bleeding control techniques during the procedure, to prevent or reduce bleeding.

For some applications, the procedure described with reference to FIG. 30 is performed using an approach that is different from the perineal approach (e.g., the abdominal approach). For some applications, techniques are performed for creating space at a location near the prostate (e.g., near the posterior portion of the prostate, as described hereinabove). Optionally, the endoscopic instrument is introduced transabdominally, while the apparatus intended to treat the prostate is introduced transperineally. For some applications, the procedure is performed using more than a single access port, and using a plurality of instruments, as described above. For some applications, the procedure may be used for treating other conditions, e.g., focal prostatic cancer.

For some applications, the apparatus and methods described herein are practiced in combination with apparatus and techniques described in (a) PCT Application PCT/IL08/01586 to Goldwasser, (b) U.S. Provisional Application 61/185,660 to Goldwasser, and/or (c) U.S. Provisional Patent Application 61/213,732 to Beyar, all of which applications are incorporated herein by reference.

It is to be understood that although some applications of the present invention are described with respect to the use of an imaging device to facilitate capsulotomy or capsulectomy, the scope of the present invention includes performing the capsulotomy or capsulectomy without such an imaging device.

It is noted that whereas some applications of the present invention are described herein with respect to the use of an endoscope, the scope of the present invention includes the use of another longitudinal surgical introduction tool, such as a trocar or a delivery catheter, in place of or in addition to an endoscope. The scope of the invention further includes the insertion of cutting devices and/or balloons without the use of an introduction tool.

It is noted that whereas some applications of the present invention are described herein with respect to treating a constriction of the prostatic urethra due to BPH, the scope of the present invention includes using the methods and apparatus described herein to treat a constriction of the urethra due to other causes, such as an obstruction of the prostatic urethra.

It is noted that all applications described herein that are not specifically described as being carried out in a transurethral fashion are performed without puncturing the urethra.

It is further noted that whereas some applications are described hereinabove with respect to incising the prostatic capsule, the scope of the present invention includes excising at least a portion of the capsule, whether by energy-based excision techniques or using a cold knife. It is still further noted that although applications are described with respect to cutting the prostate, and/or the prostatic capsule, the scope of the present invention includes performing an incision and/or an excision in the prostate capsule with or without incising or excising the prostate tissue.

The terms “cutting” and “incising,” as used in the present application and in the appended claims, are to be understood to mean cutting, incising, and/or excising.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-137. (canceled)

138. A method, comprising:
identifying a subject as suffering from a disorder of an organ of the subject; and
in response to the identifying:
inserting a shaft into the organ, not via a natural lumen of the subject;
using a laser beam emitted from a distal portion of the shaft, ablating a portion of the organ; and
cooling a vicinity of the portion of the organ by introducing a fluid to the vicinity, and, subsequently aspirating the fluid therefrom, the introduction and aspiration of the fluid being performed via the shaft, while the shaft is inside the organ.

139. The method according to claim 138, wherein the organ includes an organ selected from the group consisting of: a prostate, a liver, and a kidney, and wherein inserting the shaft into the organ comprises inserting the shaft into the selected organ.

140. The method according to claim 138, wherein inserting the shaft comprises inserting a shaft having a diameter of less than 5 mm.

141. The method according to claim 138, wherein aspirating the fluid comprises aspirating ablation products from the organ.

142. The method according to claim 138, wherein ablating the portion comprises ablating the portion without direct vision of the portion.

143. The method according to claim 138, further comprising viewing the ablation.

144. The method according to claim 143, wherein viewing the ablation comprises using an imaging modality selected from the group consisting of: abdominal ultrasound imaging, transrectal ultrasound imaging, and transurethral ultrasound imaging.

145. The method according to claim 138, wherein the organ includes the subject's prostate and wherein identifying the subject comprises identifying the subject as suffering from a disorder selected from the group consisting of: benign prostatic hyperplasia and focal prostatic cancer.

146. The method according to claim 145, further comprising incising a prostate capsule of the subject.

147. The method according to claim 145, wherein inserting the shaft comprises inserting the shaft without damaging a urethra of the subject, and wherein ablating the portion comprises ablating the portion without damaging the urethra.

148. The method according to claim 145, wherein inserting the shaft comprises inserting the shaft transperineally into the subject's prostate.

149. Apparatus for use with a cooling fluid, and for treating a subject suffering from a disorder of an organ, the apparatus comprising:
a laser device, comprising:
a shaft having a diameter of less than 5 mm,
a laser fiber disposed inside the shaft, and configured to ablate a portion of the selected organ by directing a laser beam toward the portion,
the shaft defining:
an introduction lumen configured to facilitate cooling of a vicinity of the portion by introducing the cooling fluid to the vicinity, and
an aspiration lumen configured to facilitate aspiration of the cooling fluid from the portion.

150. A method, comprising:
identifying a subject as suffering from a disorder selected from the group consisting of: benign prostatic hyperplasia and focal prostatic cancer; and
in response to the identifying:
transperineally inserting a tool into a retropubic space of the subject; and
while at least a portion of the tool is disposed in the retropubic space, performing an action with respect to a portion of a prostate of the subject using the tool, the action being selected from the group consisting of: ablation, incision, imaging and resection.

151. The method according to claim 150, wherein inserting the tool comprises inserting the tool minimally-invasively.

152. The method according to claim 150, wherein inserting the tool comprises inserting the tool without damaging a urethra of the subject.

153. The method according to claim 150, wherein performing the selected action comprises ablating the subject's prostate using a laser.

154. The method according to claim 150, wherein performing the selected action comprises incising a prostate capsule of the subject's prostate.

155. The method according to claim 150, wherein performing the selected action comprises excising a portion of the subject's prostate.

156. The method according to claim 150,
wherein the tool includes (a) an endoscope having a working channel, and (b) a treatment tool,
wherein inserting the tool into the retropubic space comprises (c) inserting the endoscope into the retropubic space and (d) inserting the treatment tool into the retropubic space via the working channel of the endoscope, and
wherein performing the selected action comprises (e) performing an action on the portion of the prostate, with the treatment tool, the action being selected from the group consisting of: ablation, incision and resection, and (f) imaging, with the endoscope, the action being performed by the treatment tool.

157. The method according to claim 150,
wherein the tool includes (a) an endoscope and (b) a treatment tool,
wherein inserting the tool into the retropubic space comprises (c) inserting the endoscope into the retropubic space via a first access port in a perineum of the subject, and (d) inserting the treatment tool into the retropubic space via a second access port in the subject's perineum, and
wherein performing the selected action comprises (e) performing an action with the treatment tool selected from the group consisting of: ablation, incision and resection, and (f) imaging the action being performed by the treatment tool with the endoscope.

158. The method according to claim 150, wherein the tool includes an endoscope, wherein inserting the tool into the retropubic space comprises inserting the endoscope into the retropubic space, and wherein performing the selected action comprises imaging the portion of the prostate with the endoscope.

159. The method according to claim 158, further comprising transperineally inserting a treatment tool into the prostate, and, while the endoscope is imaging the portion of the prostate, performing an action on the portion of the prostate with the treatment tool, the action being selected from the group consisting of: ablation, incision, and resection.

160. The method according to claim 150, further comprising enlarging the retropubic space prior to performing the selected action.

161. The method according to claim 160,
wherein the tool includes (a) a balloon, and (b) a treatment tool,
wherein inserting the tool into the retropubic space comprises:
first, inserting the balloon into the retropubic space and enlarging the retropubic space by inflating the balloon, and
subsequently, inserting the treatment tool into the retropubic space, and
wherein performing the selected action comprises performing the selected action with the treatment tool.

162. The method according to claim 160,
wherein the tool includes (a) a fluid-introduction device, and (b) a treatment tool,
wherein inserting the tool into the retropubic space comprises:
first, inserting the fluid-introduction device into the retropubic space and enlarging the retropubic space by introducing fluid directly into the retropubic space via the fluid-introduction device, and
subsequently, inserting the treatment tool into the retropubic space, and wherein performing the selected action comprises performing the selected action with the treatment tool.
